(19) United States
(12) Patent Application Publication
(10) Pub. No.: US 2023/0167477 A1
(43) Pub. Date: Jun. 1, 2023

(54) Protein Purification

(71) Applicant: Nuclera Nucleics Ltd., Cambridge (GB)

(72) Inventors: Michael Chun Hao Chen, Cambridge (GB); Gordon Ross McInroy, Cambridge (GB); Tobias William Barr Ost, Cambridge (GB)

(21) Appl. No.: 17/982,080

(22) Filed: Nov. 7, 2022

Related U.S. Application Data
(63) Continuation of application No. PCT/GB2022/052809, filed on Nov. 7, 2022.

(30) Foreign Application Priority Data
Nov. 5, 2021 (GB) 2115956.1
Jul. 14, 2022 (GB) 2210332.9
Jul. 14, 2022 (GB) 2210384.0

(51) Int. Cl. C12P 21/00; B01L 3/00; C12M 1/40; C12M 1/34; C12M 1/00

(52) U.S. Cl. CPC C12P 21/00; B01L 3/502761; C12M 21/18; C12M 41/32; C12M 47/10; B01L 2400/043; B01L 2400/0427; B01L 2200/04; B01L 2200/0652; B01L 2200/16

(57) ABSTRACT

The invention provided herein relates to methods for protein synthesis, characterisation and purification on a digital microfluidic device.

### REFERENCE TO RELATED APPLICATION

This application is a continuation application, filed under 35 U.S.C. 111(a), of International Patent Application No. PCT/GB2022/052809, filed Nov. 7, 2022, which claims foreign priority to UK Patent Application GB2115956.1, filed Nov. 5, 2021; UK Patent Application GB2210332.9, filed Jul. 14, 2022; and UK Patent Application GB2210384.0, filed Jul. 14, 2022, the entire contents of each of which, including drawings and any sequence listings, are incorporated herein by reference.

### REFERENCE TO THE SEQUENCE LISTING

The instant application contains a Sequence Listing XML file, which has been submitted electronically in XML format and is hereby incorporated by reference in its entirety. Said XML copy, created on Feb. 21, 2023, is named 135815-01301.xml and is 51,040 bytes in size.

### FIELD OF THE INVENTION

Provided herein are methods for purification of proteins or other biomolecules on digital microfluidic (DMF) device arrays.

### BACKGROUND

Proteins are biological macromolecules that maintain the structural and functional integrity of the cell, and many diseases are associated with protein malfunction. Protein purification is a fundamental step for analysing individual proteins and protein complexes and identifying interactions with other proteins, DNA or RNA. A variety of protein purification strategies exist to address desired scale, throughput and downstream applications. However, protein production can be challenging for many reasons. One major challenge is finding a suitable expression system, for example sourced from mammalian, bacterial, fungal, or plant cells. This can take months of work.

Purification of proteins using functionalized magnetic beads (superparamagnetic particles, SPMP) is well known in the literature. One common method is to express a protein with a particularly tag sequence, for example a His tag (typically 6× histidine amino acids (SEQ ID NO: 10) at either the N- or C-terminus) and isolate this protein from lysed cells using Ni-NTASPMP. Another common method is to express a protein with a Strep or Strep II tag (WSHPQFEK (SEQ ID NO: 27) or AWAHPQPGG (SEQ ID NO: 28) amino acid tags at N- or C-terminus) and isolate this protein from lysed cells using Streptavidin (or a related derivative) SPMP. Many types of tag sequences or binding moieties are known.

Electrowetting is the modification of the wetting properties of a surface (which is typically hydrophobic) with an applied electric field. Microfluidic devices for manipulating droplets or magnetic beads based on electrowetting have been extensively described. In the case of droplets in channels this can be achieved by causing the droplets, for example in the presence of an immiscible carrier fluid, to travel through a microfluidic channel defined by the walls of a cartridge or microfluidic tubing. Embedded in the walls of the cartridge or tubing are electrodes covered with a dielectric layer each of which are connected to an A/C biasing circuit capable of being switched on and off rapidly at intervals to modify the electrowetting field characteristics of the layer. This gives rise to the ability to steer the droplet along a given path.

As an alternative to microfluidic channel systems, droplets can also be generated and manipulated on planar surfaces using digital microfluidics (DMF). In contrast to channel based microfluidics, DMF utilizes alternating currents on an electrode array for moving fluid on the surface of the array. Liquids can thus be moved on an open-plan device by electrowetting. Digital microfluidics allows precise control over the droplet movements including droplet fusion and separation.

Cell-free protein synthesis, also known as in vitro protein synthesis or CFPS, is the production of peptides or proteins using biological machinery in a cell-free system, that is, without the use of living cells. The in vitro protein synthesis environment is not constrained within a cell wall or limited by conditions necessary to maintain cell viability, and enables the rapid production of any desired protein from a nucleic acid template, usually plasmid DNA or RNA from an in vitro transcription. CFPS has been known for decades, and many commercial systems are available. Cell-free protein synthesis encompasses systems based on crude lysate (Cold Spring Harb Perspect Biol. 2016 December; 8(12): a023853) and systems based on reconstituted, purified molecular reagents, such as the PURE system for protein production (Methods Mol Biol. 2014; 1118: 275-284). CFPS requires significant concentrations of biomacromolecules, including DNA, RNA, proteins, polysaccharides, molecular crowding agents, and more (Febs Letters 2013, 2, 58, 261-268).

To date, digital microfluidics, electrowetting-on-dielectric (EWoD), and electrokinesis in general have only found limited uses in cell-free biological-based applications, mostly due to biofouling, where biological components such as proteins, nucleic acids, crude cell extracts and other bioproducts adsorb and/or denature to hydrophobic surfaces. Biofouling is well known in the art to limit the ability of EWoD devices to manipulate droplets containing biomacromolecules. Wheeler and colleagues report that the maximum actuation time for droplets on EWoD devices containing biological media is 30 min before biofouling inhibits EWoD-based droplet actuation (Langmuir 2011, 27, 13, 8586-8594).

WO2020/178604 describes a method of oligonucleotide synthesis carried out in a single device. The method synthesis oligonucleotides and assembles them into contiguous strands.

WO2018/053174 describes devices and methods for sample analysis where nucleic acid samples are captured into wells on a device.

Digital microfluidics can be carried out in an air-filled system where the liquid drops are manipulated on the surface in air. However, at elevated temperatures or over prolonged periods, the volatile aqueous droplets simply dry onto the surface by evaporation. This issue is compounded by the high surface area to volume ratio of nanoliter and microliter sized drops. Hence air-filled systems are generally not suitable for protein expression where the temperature of the system needs to be maintained at a temperature suitable for enzyme activity and the duration of the synthesis needs to be prolonged for synthesized proteins levels to be detectable. Digital microfluidics is carried out on an open planar surface without the need for physical features such as wells.

### SUMMARY OF THE INVENTION

The invention relates to the synthesis, characterisation and purification of biopolymers on a digital microfluidic (DMF) device. Described are methods for biopolymer synthesis and purification.

Following protein expression on digital microfluidic devices, a system of purification of the resultant biomolecules is required. Due to the challenges of manipulating protein on digital microfluidic devices (including biofouling, interaction with hydrophobic surfaces, interaction with electric fields, aqueous/oil interfaces) protein purification is particularly challenging.

Disclosed herein is a method for polymer synthesis and purification on a digital microfluidic device comprising

a. taking a digital microfluidic device having a planar array of electrodes;
b. performing synthesis of a polymer of interest in droplets on the device, wherein the polymers contain a binding moiety;
c. immobilising the polymers via the binding moiety;
d. removing the immobilised polymers from the droplets using the electrodes to move the droplets away from the immobilised polymers; and
e. optionally releasing the immobilised polymers into further droplets.

Disclosed is a method comprising

a. taking a digital microfluidic device having a planar array of electrodes;
b. synthesising a protein of interest having a binding tag in droplets on the device;
c. capturing the proteins via the binding tags, thereby immobilising the proteins;
d. moving the droplets using the electrodes, thereby removing the synthesised proteins from the droplet;
e. optionally washing the immobilised proteins; and
f. optionally releasing the proteins into further droplets.

Disclosed is a method comprising

a. taking a digital microfluidic device having a planar array of electrodes;
b. using a variety of different conditions to synthesise a protein of interest having a tag in one or more droplets on the device, thereby identifying the optimal conditions for the expression of soluble protein having the tag;
c. removing the droplets from the device;
d. using the optimal conditions to synthesise a protein of interest in a further population of droplets on the device;
e. capturing the proteins via affinity to magnetic beads, thereby immobilising the proteins;
f. moving the droplets using the electrodes, thereby removing the immobilised synthesised proteins from the droplet;
g. optionally washing the immobilised proteins; and
h. removing the proteins from the device, either by releasing the proteins into further droplets and removing the protein droplets from the device or by removing the magnetic beads from the device.

Also disclosed is a method comprising

a. taking a digital microfluidic device having a planar array of electrodes;
b. performing nucleic acid synthesis in droplets on the device, wherein the full-length nucleic acid is coupled to a binding moiety;
c. immobilising the full length nucleic acids via the binding moiety;
d. removing the full-length nucleic acids from the droplets using the electrodes to move the droplets;
e. optionally washing the immobilised nucleic acids; and
f. optionally releasing the full-length nucleic acids into further droplets.

For all methods disclosed herein the digital microfluidic device may comprise:

a. a first substrate having a matrix of electrodes, wherein each of the matrix electrodes is coupled to a thin film transistor, and wherein the matrix electrodes are overcoated with a functional coating comprising:
i. a dielectric layer in contact with the matrix electrodes,
ii. an optional conformal layer in contact with the dielectric layer, and
iii. a hydrophobic layer in contact with the matrix electrodes or conformal layer;
   b. a second substrate comprising a top electrode;
   c. a spacer disposed between the first substrate and the second substrate and defining an electrokinetic workspace; and
   d. a voltage source operatively coupled to the matrix electrodes.

For all methods disclosed herein the digital microfluidic device may comprise:

a. a first substrate having a matrix of electrodes, wherein each of the matrix electrodes is coupled to a thin film transistor, and wherein the matrix electrodes are overcoated with a functional coating comprising:
i. a dielectric layer in contact with the matrix electrodes,
ii. an optional conformal layer in contact with the dielectric layer, and
iii. a hydrophobic layer in contact with the matrix electrodes or conformal layer;
   b. a second substrate comprising a top electrode;
   c. a spacer disposed between the first substrate and the second substrate and defining an electrokinetic workspace; and
   d. a voltage source operatively coupled to the matrix electrodes.

### BRIEF DESCRIPTION OF THE FIGURES

FIG. 1: A schematic showing polymer synthesis and purification. (A) monomer components for the polymer shown as rectangles, other necessary synthesis components shown as hexagons and ellipses; (B) synthesised polymer with a binding tag in presence of other components; (C) solid support with binding partner for binding tag is introduced to the system; (D) the polymer is immobilized on the solid support via the binding tag and the supernatant removed—some unwanted components may remain; (E) optional addition washes remove all unwanted components; (F) the polymer may optionally be removed from the solid support and thus released into solution.

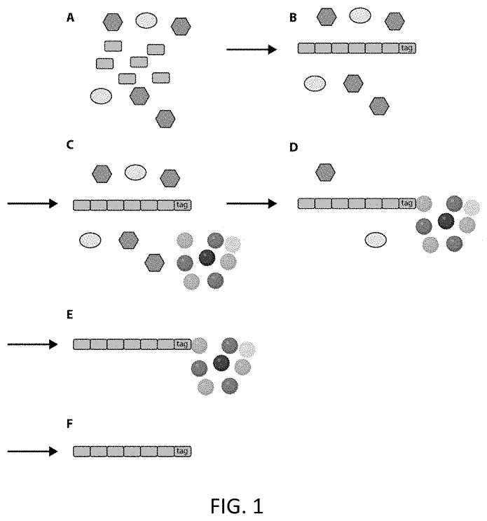

FIGS. 2A-2J: A schematic showing the process shown in FIG. 1 on a digital microfluidic device, using cell-free protein synthesis as an example. The bounding box in each step shows the same sub-section of a larger digital microfluidic array and is for clarity only. (FIG. 2A) A 4×4 droplet containing cell-free lysate and a linear expression template coding for Protein X bearing a Strep II binding tag; (FIG. 2B) the same droplet now containing cell-free lysate, linear expression template and newly expressed Protein X; (FIG. 2C) the droplet from step (FIG. 2B) alongside a 3×3 droplet containing superparamagnetic particles (SPMPs) coated with streptavidin or Strep-Tactin; (FIG. 2D) the reagent and particle drop are merged and incubated to allow binding tag and binding partner to interact—note the size has increased due to merging of droplets creating a single 5×5 droplet; (FIG. 2E) a magnet is brought into proximity with the droplet to pellet the SPMPs; (FIG. 2F) the supernatant droplet is removed from the pelleted SPMPs/immobilised protein X; (FIG. 2G) an elution droplet is brought over the pelleted particles, for instance containing desthiobiotin; (FIG. 2H) the magnet is removed and the SPMPs are resuspended in the elution droplet; the polymer is released into solution; (FIG. 2I) the magnet is engaged; (FIG. 2J) the purified protein in solution is moved away.

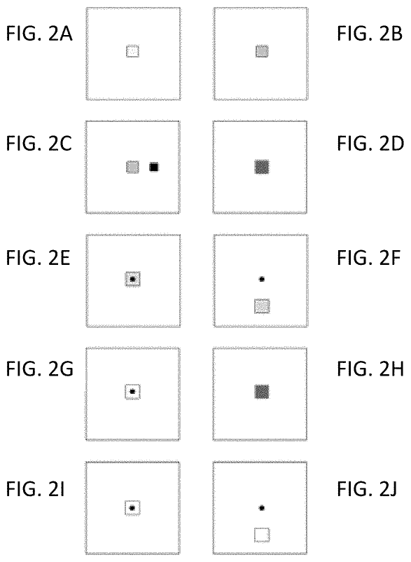

FIG. 3: A schematic showing protein screening and scale-up on a digital microfluidic device having a planar array of electrodes. Proteins are screened using different conditions such as varying nucleic acid template lengths (optionally to include different solubility factors, binding tags etc), protein expression compositions, buffers etc. The winning conditions can be identified using a variety of selection criteria to determine the level of soluble protein produced. For example the protein can be expressed having a binding tag that can be measured. The binding tag optionally can be a sub-component of a fluorescent protein, in which case the remaining sub-components of the fluorescent protein can be present as a detector. Upon expression of the tag, the fluorescent protein assembles and the fluorescence signal gives a measure of soluble protein present in each droplet. The highest fluorescence is obtained in the best conditions for expression of the protein of interest. Once the best conditions are identified and stored, the device can be transitioned and used again to scale up the protein synthesis using the identified optimal conditions. The screening population can be removed from the device or moved to a waste section in a separate part of the device. A further population of droplets is prepared using the best nucleic template and expression compositions and the protein expressed in the same conditions in a population of droplets on the device. Following expression the proteins are separated from the expression reagents via binding to a solid support using the binding tag. The solid support may be magnetic beads. The expressed proteins having binding tags are therefore separated from all the expression reagents not having binding tags. The beads may be immobilised on the device. The immobilised beads may be washed with one or more further droplet exchanges on the device to remove any non-bound material. The beads may be held immobilised on the device whilst liquid, in the form of droplets, is added and removed. The beads may be released from the device and removed. Once removed the protein may be released from the device. Alternatively the proteins may be released from the beads and harvested from the device. The release may comprise a washing step to release the binding tag, or a chemical cleavage, which may occur on the device such that the beads are retained on the device, or off the device if the beads are collected.

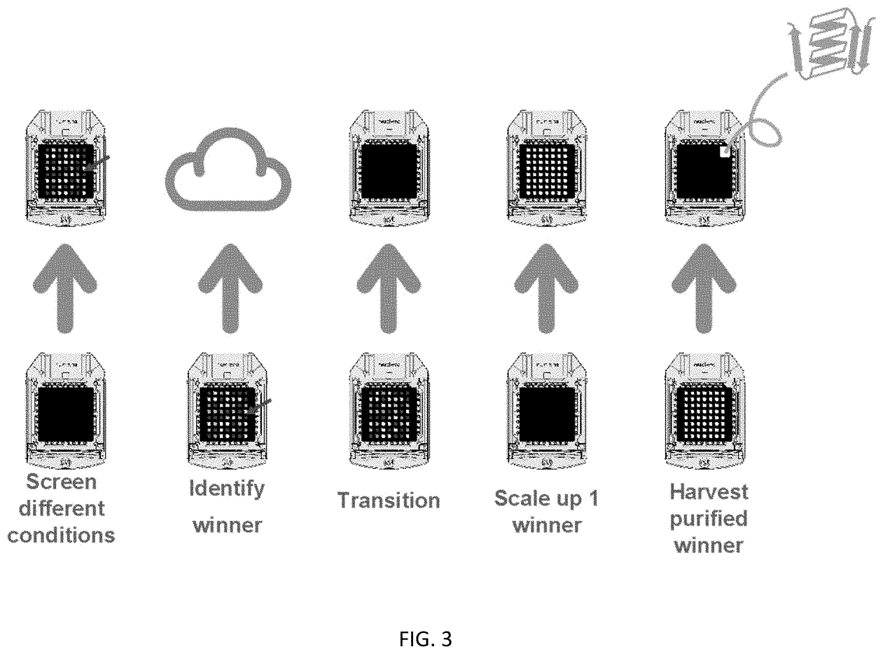

FIG. 4: shows the purification workflow. Magnetic particles are added to nL samples droplets and bind to the expressed target protein. The magnetic particles are captured and the remainder of the sample is washed away. The target protein is released from the magnetic particles into a further droplet for further analysis.

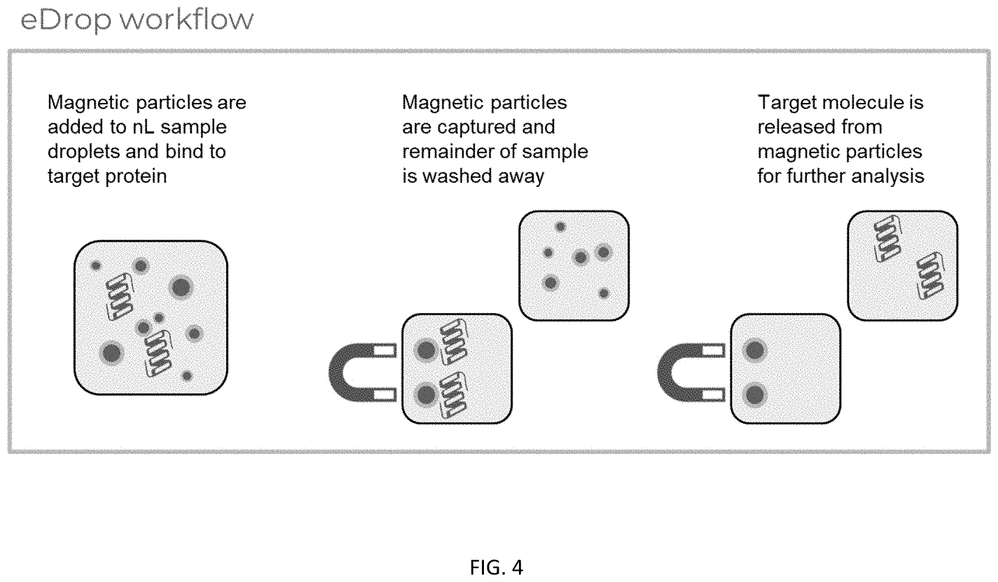

FIG. 5: shows droplets of liquid containing beads being manipulated on a digital microfluidic device having a planar array of electrodes. An array of droplets having beads (white) is merged with an array of droplets containing samples (Frame 1). The beads can be immobilised and the liquid removed (Frame 2). Frame 3 shows a fluorescence image of eluted protein, i.e. the protein (fluorescent) is eluted from the beads. The protein can be eluted from the device. Frame 4 shows a SDS-PAGE gel of the eluted proteins. The droplets were merged in groups of 4 droplets, but could be collected in any combination of droplets on the device. The SDS page gel shows that the eluted protein is a single band. The same tag can be used to both detect expression and purify the expressed material. Alternatively different tags can be used. For example the detection tag may be a fluorescent protein sub-component such as GFP₁₁, and the binding tag may be an affinity tag such as polyhistidine.

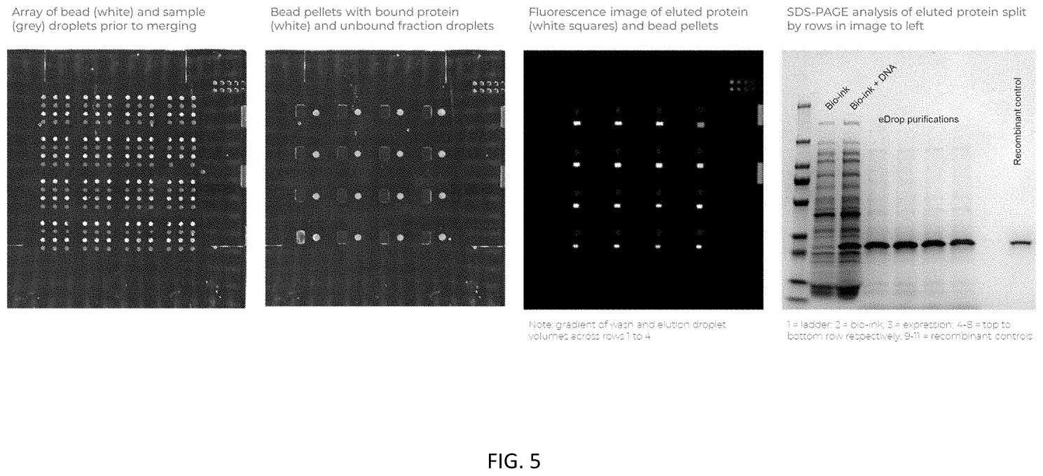

FIG. 6: shows the workflow for bead handling. The crude material can be missed with magnetic particles on the device. The particles can be held in place and washed to elute unbound material. The beads can be further washed to aid purification. The purified material can be eluted from the beads and removed from the device.

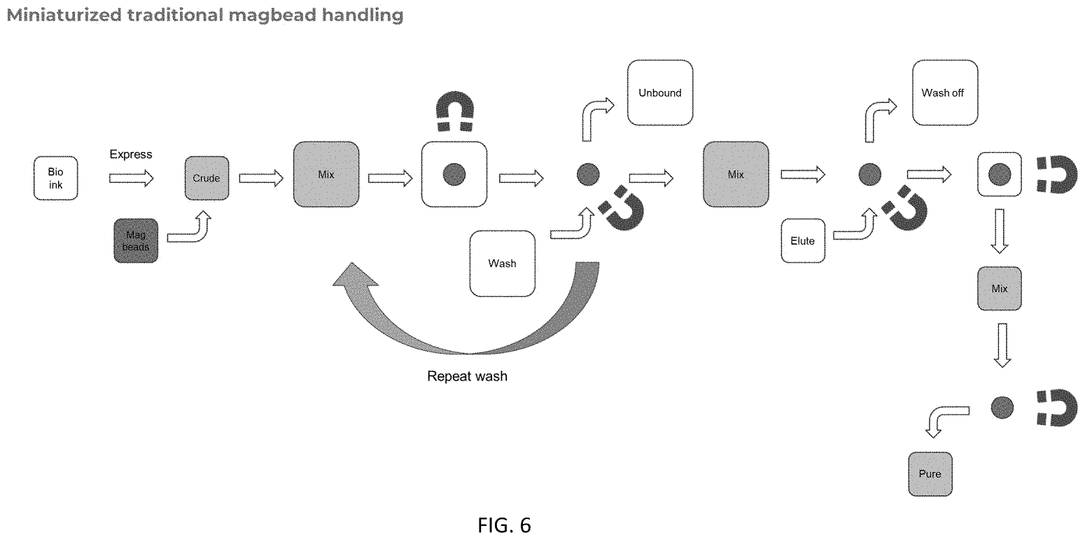

FIG. 7: shows fluorescence intensity images of 48 expression conditions (12 constructs and 4 expression reagents) and controls on an EWoD device.

FIG. 8: shows an SDS-PAGE image of on device His-tag purification of ccGFP expressed in an E coli lysate.

FIG. 9: shows an SDS-PAGE image of on device His-tag purification of ccGFP expressed in a reconstituted transcription/translation system.

FIG. 10: shows an SDS-PAGE image of on-device Strep-tag purification of ccGFP expressed in a reconstituted transcription/translation system.

### DETAILED DESCRIPTION OF THE INVENTION

Disclosed herein is a method for biopolymer synthesis and purification on a digital microfluidic device comprising

a. taking a digital microfluidic device having a planar array of electrodes;
b. performing synthesis of a polymer of interest in droplets on the device, wherein the biopolymers contain a binding moiety;
c. immobilising the biopolymers via the binding moiety;
d. removing the immobilised biopolymers from the droplets using the electrodes to move the droplets away from the immobilised biopolymers; and
e. optionally releasing the immobilised biopolymers into further droplets.

Disclosed herein is a method comprising

a. taking a digital microfluidic device having a planar array of electrodes;
b. using a variety of different conditions to synthesise a protein of interest having a tag in one or more droplets on the device, thereby identifying the optimal conditions for the expression of soluble protein having the tag;
c. removing the droplets from the device;
d. using the optimal conditions to synthesise a protein of interest having a binding tag in a further population of droplets on the device;
e. capturing the proteins via affinity to magnetic beads, thereby immobilising the proteins;
f. moving the droplets using the electrodes, thereby removing the immobilised synthesised proteins from the droplet;
g. optionally washing the immobilised proteins; and
h. removing the proteins from the device, either by releasing the proteins into further droplets and removing the protein droplets from the device or by removing the magnetic beads from the device.

Disclosed is a method for protein purification comprising

a. taking a digital microfluidic device having a planar array of electrodes;
b. using a variety of different conditions to purify a protein of interest having a tag in one or more droplets on the device, thereby identifying the optimal conditions for the purification of soluble protein having the tag;
c. removing the droplets from the device;
d. using the optimal conditions to purify a protein of interest in a further population of droplets on the device;
e. capturing the proteins via affinity to magnetic beads, thereby immobilising the proteins;
f. moving the droplets using the electrodes, thereby removing the immobilised synthesised proteins from the droplet;
g. optionally washing the immobilised proteins; and
h. removing the proteins from the device, either by releasing the proteins into further droplets and removing the protein droplets from the device or by removing the magnetic beads from the device.

Disclosed is a method comprising

a. taking a digital microfluidic device having a planar array of electrodes;
b. using a variety of different conditions to synthesise a protein of interest having a tag in one or more droplets on the device, thereby identifying the optimal conditions for the expression of soluble protein having the tag;
c. removing the droplets from the device;
d. using the optimal conditions to synthesise a protein of interest having a binding tag in a further population of droplets on the device;
e. capturing the proteins via affinity to magnetic beads, thereby immobilising the proteins;
f. moving the droplets using the electrodes, thereby removing the immobilised synthesised proteins from the droplet;
g. washing the immobilised proteins;
h. removing the proteins from the beads by releasing the proteins into further droplets; and
i. measuring the total concentration of protein and the yield of soluble protein in a portion of the droplets to determine the soluble yield and purity of the synthesised protein.

This screening workflow enables users to rapidly screen different expression systems in the form of cell free lysates within a day on a digital microfluidic (DMF) device. Having identified an optimal expression system, a scientist typically wants to obtain small quantities of protein to perform initial tests, for instance to verify protein molecular weight, solubility, and function (whether activity or binding affinity). To perform these tests a pure protein is typically required, hence there is a need for a method to separate a protein of interest from a complex mixture containing other proteins, nucleic acids, and other cellular components on a digital microfluidic device.

The digital microfluidic device used in the methods disclosed herein can be an electrowetting on dielectric (EWoD) device. More specifically, the device can be an AM-EWoD device.

The polymers or biopolymers which are synthesised in the methods disclosed can be cellulose and starch, proteins and peptides, DNA and RNA. The polymer may contain phosphodiester, amide or glycosidic bonds. The polymer can be made of naturally occurring or non-naturally occurring monomers or a mixture thereof. The polymer can be made by coupling monomers via the formation of phosphodiester, amide or glycosidic bonds.

The purification is based on a property of the expressed protein. The expressed protein can be purified via immobilisation. The immobilisation can be based on for example charge, size, hydrophobic interactions or based on specific sequence interactions such as binding of Protein A. The purification may be a multi-stage purification, for example binding to a first bead and then binding to a second bead.

The protein can be expressed with a binding tag. The tag binding moiety can be incorporated during biopolymer synthesis or can be attached via conjugation after biopolymer synthesis. The binding tags may be attached via coupling to a functionalizable moiety on the polymer. The conjugation can be for example via chemical attachment such as for example via click chemistry using an azide/alkyne. The conjugation can be performed for example using a specific amino acid, which could be a natural or unnatural amino acid. The expressed translated protein may have an amino acid allowing protein modification post translation, for example a cysteine or lysine which can be chemically reacted with the tag sequence.

Immobilisation can be performed using magnetic beads. Another example could be the use of a polymer patch. The polymer patch may be functionalised with NTA (to bind Ni2+, Tb3+) or to modified to immobilize streptavidin, thereby obviating the need to magnetize particles. The patch could be formed and functionalized in situ on the DMF and used as a purification zone to (and from) which expressed, tagged polymers/proteins could be bound, washed and eluted. Biocompatible polymers such as polyacrylamide hydrogels can be used for this purpose and can be fabricated in DMF cell gaps.

The binding moiety can be a region of amino acid/peptide sequence. The affinity binding site can be a region of amino acid/peptide sequences specific to a particular antibody. For example the binding moiety can be selected from the list of exemplary peptide affinity binding sites below:

Alfa-tag
(SEQ ID NO: 1)
(SRLEEELRRRLTE)
Avi-tag
(SEQ ID NO: 2)
(GLNDIFEAQKIEWHE)
C-tag
(SEQ ID NO: 3)
(EPEA)
Calmodulin-tag
(SEQ ID NO: 4)
(KRRWKKNFIAVSAANRFKKISSSGAL)
Dogtag
(SEQ ID NO: 5)
(DIPATYEFTDGKHYITNEPIPPK)
E-tag
(SEQ ID NO: 6)
(GAPVPYPDPLEPR)
FLAG
(SEQ ID NO: 7)
(DYKDDDDK)
G4T
(SEQ ID NO: 8)
(EELLSKNYHLENEVARLKK)
HA
(SEQ ID NO: 9)
(YPYDVPDYA)
His
(SEQ ID NO: 10)
(HHHHHH)
Isopeptag
(SEQ ID NO: 11)
(TDKDMTITFTNKKDAE)
lanthanide binding tag
(SEQ ID NO: 12)
(FIDTNNDGWIEGDELLLEEG)
Myc
(SEQ ID NO: 13)
(EQKLISEEDL)
NE-Tag
(SEQ ID NO: 14)
(TKENPRSNQEESYDDNES)
Poly Glutamate-tag
(SEQ ID NO: 15)
(EEEEEEE)
Poly Arginine-tag
(SEQ ID NO: 16)
(RRRRRRR)
Rho1D4-tag
(SEQ ID NO: 17)
(TETSQVAPA)
SBP-tag
(SEQ ID NO: 18)
(MDEKTTGWRGGHVVEGLAGELEQLRARLEHHPQGQREP)
Sdytag
(SEQ ID NO: 19)
(DPIVMIDNDKPIT)
SH3
(SEQ ID NO: 20)
(STVPVAPPRRRRG)
Snooptag
(SEQ ID NO: 21)
(KLGDIEFIKVNK)
Softag 1
(SEQ ID NO: 22)
(SLAELLNAGLGGS)
Softag 3
(SEQ ID NO: 23)
(TQDPSRVG)
Spot-tag
(SEQ ID NO: 24)
(PDRVRAVSHWSS)
Spytag
(SEQ ID NO: 25)
(AHIVMVDAYKPTK)
S-tag
(SEQ ID NO: 26)
(KETAAAKFERQHMDS)
Strep-tag
(SEQ ID NO: 27)
(WSHPQFEK)
Strep-tag II
(SEQ ID NO: 28)
(AWAHPQPGG)
T7tag
(SEQ ID NO: 29)
(MASMTGGQQMG)
TC-tag
(SEQ ID NO: 30)
(EVHTNQDPLD)
Ty-tag
(SEQ ID NO: 31)
(CCPGCC)
VSV-tag
(SEQ ID NO: 32)
(YTDIEMNRLGK)
Xpress-tag
(SEQ ID NO: 33)
(DLYDDDDK)

A single “tag” can be used for detection AND for purification to prevent the encoded payload in the expression cassette becoming too large or to prevent multiple tags from interfering with the function of the biomolecule. In particular examples the tag can also be used to detect the presence of the biopolymer. Thus the tag can be used for the dual purpose of both detection and purification. This is advantageous in preventing the biopolymer becoming too large. In the case of proteins, addition of too much “exogenous” protein fused to either the N or C terminus of the protein can sometimes change the activity or function of the protein.

The binding moiety tag can be a sub-component of a fluorescent protein. Thus the fully assembled protein becomes fluorescent. For example if the immobilised material contains GFP₁₋₁₀ and the tag contains a GFP₁₁ peptide, complementation forms immobilised fluorescent GFP, allowing simultaneous monitoring and purification. The immobilised material can be washed and then eluted by disrupting the complemented split GFP, e.g. through the use of salt or temperature.

The method may comprise:

a. taking a digital microfluidic device having a planar array of electrodes;
b. synthesising a protein of interest having a binding tag in droplets on the device;
c. capturing the proteins via the binding tags, thereby immobilising the proteins;
d. moving the droplets using the electrodes, thereby removing the synthesised proteins from the droplet;
e. optionally washing the immobilised proteins; and
f. optionally releasing the proteins into further droplets.

The immobilising may be via a secondary interaction with another protein which is, or can be, immobilised. Thus the method may comprise

a. taking a digital microfluidic device having a planar array of electrodes;
b. synthesising a protein of interest having a binding tag in droplets on the device;
c. capturing the proteins via the binding tags, thereby immobilising the proteins, wherein the immobilisation of the binding tag is via binding to another protein;
d. moving the droplets using the electrodes, thereby removing the synthesised proteins from the droplet;
e. optionally washing the immobilised proteins; and
f. optionally releasing the proteins into further droplets.

An exemplary method may comprise synthesising a protein of interest having a binding tag where the binding tag is a component of a fluorescent protein. For example the binding tag may be GFP₁₁. The synthesised protein may be captured via immobilisation to a protein comprising GFP₁₋₁₀, which may have a further tag (such as HIS or Strep). Thus the assembled GFP₁₋₁₁ protein along with the synthesised protein is captured via binding of the whole complex. The immobilised protein complex can be washed etc as described.

Release of the synthesied protein can be achieved in several ways. Disrupting binding of the complex to the solid support will release the assembled GFP₁₋₁₁ protein along with the synthesised protein. Alternatively the protein interaction forming the GFP₁₋₁₁ protein may be disrupted, breaking away the synthesised GFP₁₁ protein leaving the GFP₁₋₁₀ immobilised. Alternatively the protein sequence may be cleaved, for example using a protease, thereby separating a protein of interest from the immobilised GFP₁₋₁₁ protein. Such cleavage conditions may be preferred in order to remove unwanted tag sequences from a protein of interest.

Disclosed is a method for synthesizing a protein of interest (POI) comprising:

a. taking a digital microfluidic device having a planar array of electrodes;
b. synthesising a protein of interest having a binding tag in droplets on the device, wherein the binding tag is a sub-component of a fluorescent protein;
c. capturing the POI via the binding tags to form a fluorescent protein, and capturing the fluorescent protein, thereby immobilising the POI,
d. moving the droplets using the electrodes, thereby removing the synthesised fluorescent proteins from the droplet;
e. optionally washing the immobilised fluorescent proteins; and
f. releasing the proteins of interest into further droplets.

In the methods described herein, the binding tag may be GFP₁₁ such as ccGFP₁₁. The GFP₁₁ may be captured via GFP₁₋₁₀ having a moiety such as HIS or Strep or any binding tag listed herein, and thereby immobilised. The immobilised proteinaceous material may be washed using further droplets. The protein of interest may be released using a protease that cleaves the GFP₁₁ from the POI, in which case the fluorescent protein complex remains immobilised.

Disclosed is a method for synthesizing a protein of interest (POI) comprising:

a. taking a digital microfluidic device having a planar array of electrodes;
b. synthesising a protein of interest having a GFP₁₁ binding tag and a protease cleavage site in droplets on the device;
c. capturing the POI via the binding tags to form GFP₁₋₁₁, and capturing the GFP₁₋₁₁, thereby immobilising the POI,
d. moving the droplets using the electrodes, thereby removing the synthesised fluorescent proteins from the droplet;
e. optionally washing the immobilised fluorescent proteins; and
f. releasing the proteins of interest into further droplets using a protease.

The method may comprise

a. taking a digital microfluidic device having a planar array of electrodes;
b. performing nucleic acid synthesis in droplets on the device, wherein the full-length nucleic acid is coupled to a binding moiety;
c. immobilising the full length nucleic acids via the binding moiety;
d. removing the full-length nucleic acids from the droplets using the electrodes to move the droplets;
e. optionally washing the immobilised nucleic acids; and
f. releasing the full-length nucleic acids into further droplets.

The binding moiety may include a small molecule affinity tag such as biotin. The binding moiety may include a particular sequence of nucleic acids. The binding moiety may include a reactive chemical moiety such as a thiol, amine, azide or alkyne.

The release of the immobilised biomolecules may be via cleavage of the tag or via disruption of binding of the tag to the support. The disruption may be via a change of buffer. The buffer may contain an agent which disrupts binding, for example imidazole for His/Ni NTA or biotin/desthiobiotin for Strep tag/Streptavidin. The disruption may be via a change in temperature. The cleavage of the tag my involve cleavage of the amino acid sequence using a protease. The protease may be a serine, threonine or cysteine protease. The protease may be for example TEV protease or 3C protease.

The droplets can be manipulated by electrokinesis in order to effect and improve protein purification. The droplet can be moved using any means of electrokinesis. The droplet can be moved using electrowetting on dielectric (EWoD). The electrical signal on the EWoD or optical EWoD device can be delivered through segmented electrodes, active-matrix thin-film transistors, or digital micromirrors.

The droplets can be formed before entering the microfluidic device and flowed into the device. Alternatively, the droplets can be merged on the device.

The cell-free expression of peptides or proteins can use a cell lysate having the reagents to enable protein expression. Common components of a cell-free reaction include an energy source, a supply of amino acids, cofactors such as magnesium, and the relevant enzymes. A cell extract is obtained by lysing the cell of interest and removing the cell walls, DNA genome, and other debris by centrifugation. The remains are the cell machinery including ribosomes, aminoacyl-tRNA synthetases, translation initiation and elongation factors, nucleases, etc. Once a suitable nucleic acid template is added, the nucleic acid template can be expressed as a peptide or protein using the cell derived expression machinery.

Any particular nucleic acid template can be expressed using the system described herein. Three types of nucleic acid templates used in cell free protein synthesis (CFPS) include plasmids, linear expression templates (LETs), and mRNA. Plasmids are circular templates, which can be produced either in cells or synthetically. LETs can be made via PCR. mRNA can be produced through in vitro transcription systems. The methods can use a single nucleic acid template per droplet. The methods can use multiple nucleic acid templates per drop. The methods can use multiple droplets having a different nucleic acid template per droplet.

An energy source is an important part of a cell-free reaction. Usually, a separate mixture containing the needed energy source, along with a supply of amino acids, is added to the extract for the reaction. Common sources are phosphoenolpyruvate, acetyl phosphate, and creatine phosphate. The energy source can be replenished during the expression process by adding further reagents to the droplet during the process.

The cell-free extract having the components for protein expression includes everything required for protein expression apart from the nucleic acid template. Thus the term includes all the relevant ribosomes, enzymes, initiation factors, nucleotide monomers, amino acid monomers, metal ions and energy sources. Once the nucleic acid template is added, protein expression is initiated without further reagents being required.

Thus the cell-lysate can be supplemented with additional reagents prior to the template being added. The cell-free extract having the components for protein expression would typically be produced as a bulk reagent or ‘master mix’ which can be formulated into many identical droplets prior to the distinct template being separately added to separate droplets. Common cell extracts in use today are made from E. coli (ECE), rabbit reticulocytes (RRL), wheat germ (WGE), insect cells (ICE) and Yeast Kluyveromyces (the D2P system). All of these extracts are commercially available.

Rather than originating from a cell extract, the cell-free system can be assembled from the required reagents. Systems based on reconstituted, purified molecular reagents are commercially available, for example the PURE system for protein production, and can be used as supplied. The PURE system is composed of all the enzymes that are involved in transcription and translation, as well as highly purified 70S ribosomes. The protein synthesis reaction of the PURE system lacks proteases and ribonucleases, which are often present as undesired molecules in cell extracts.

The use of a population of droplets having different components allows the rapid screening of a variety of variable factors to identify optimal conditions for expression of a desired proteins. The protein can be contained with a sequence having other amino acid domains, for example solubility factors or binding tags.

Protein sequences disclosed herein may be attached to further elements to improve solubility. The variant may be attached to one or more solubility enhancing sequences. The solubility enhancing sequence may be a peptide sequence or a naturally occurring sequence. The solubility enhancing sequence may be selected from for example maltose binding protein (MBP), Small Ubiquitin-like Modifier (SUMO), Glutathione S-transferase (GST) or thioredoxin (TRX). The tags may be attached to either the C or N terminus. Any example of a solubility enhancer may be used. A list of possible proteins is shown below. Any one or more sequence(s) selected from the list below may be chosen:

| | |
|---|---|
| Glutathione S-Transferase | GST |
| Small Ubiquitin-like Modifier | SUMO |
| Maltose Binding Protein | MBP |
| Fasciola hepatica 8 kDa antigen | FH8 |
| Thioredoxin | TRX |
| Solubility Enhancing Ubiquitous Tag | SNUT |
| Seventeen kilodalton protein | SKP |
| Monomeric bacteriophage T7 orc protein | MOCR |
| E coli secreted protein A | ESPA |
| N-utilization substance | NusA |
| IgG domain B1 of Protein G | GB1 |
| IgG repeat domain ZZ of Protein A | ZZ |
| Mutated dehalogenase | HaloTag |
| Phage T7 protein kinase | T7PK |
| E. coli trypsin inhibitor | Ecotin |
| Calcium-binding protein | CaBP |
| Stress-response arsenate reductase | ArsC |
| N-terminal fragment of translation initiation factor IF2 | IF2-domain 1 |
| Stress-response protein | RpoA |
| Stress-response protein | SlyD |
| Stress-response protein | Tsf |
| Stress-response protein | RpoS |
| Stress-response protein | PotD |
| Stress-response protein | Crr |
| E. coli acidic protein | msyB |
| E. coli acidic protein | yjgD |
| E. coli acidic protein | rpoD |

Any fluorescent protein may be used. The fluorescent protein may be sfGFP, GFP, eGFP, ccGFP, deGFP, frGFP, eYFP, eBFP, eCFP, Citrine, Venus, Cerulean, Dronpa, DsRED, mKate, mCherry, mRFP, FAST, SmURFP, miRFP670nano. For example the peptide tag may be GFP₁₁ and the further polypeptide GFP₁₋₁₀. The peptide tag may be one component of sfCherry. The peptide tag may be sfCherry₁₁ and the further polypeptide sfCherry₁₋₁₀. The peptide tag may be CFAST₁₁ or CFAST₁₀ and the further polypeptide CFAST in the presence of a hydroxybenzylidene rhodanine analog. The peptide tag may be ccGFP₁₁ and the further polypeptide ccGFP₁₋₁₀.

The fluorescent protein may be GFP. The fluorescent protein may be sfGFP. The fluorescent protein may be ccGFP.

The protein may be assembled and thereby become fluorescent as a result of the expressed protein binding with the binding partner. The affinity interaction results in the two sub-components of the fluorescent protein being near enough to each other to bind and induce fluorescence.

The complementary GFP₁₁ peptide amino acid sequence tag could be the following:

1.
(SEQ ID NO: 34)
KRDHMVLLEFVTAAGITGT
2.
(SEQ ID NO: 35)
KRDHMVLHEFVTAAGITGT
3.
(SEQ ID NO: 36)
KRDHMVLHESVNAAGIT
4.
(SEQ ID NO: 37)
RDHMVLHEYVNAAGIT
5.
(SEQ ID NO: 38)
GDAVQIQEHAVAKYFTV
6.
(SEQ ID NO: 39)
GDTVQLQEHAVAKYFTV
7.
(SEQ ID NO: 40)
GETIQLQEHAVAKYFTE

Properties of the expressed protein may be characterised on the device. An initial screen may be based on the level of soluble expression by measuring fluorescence formed on complementation of a detector with the expressed sequence. The protein may remain fluorescent during immobilisation, at which point the level of purification can be determined.

Further assays can be used to determine the total amount of protein present in a droplet. For example a whole protein determination assay such as the Bradford assay, which is a colorimetric protein assay based on an absorbance shift of the dye Coomassie. The assay can be performed in the same droplet or in parallel droplets. Thus droplets can be expressed in parallel, with one or more being used to measure the binding of soluble protein and different droplets used to measure total protein bound to the bead. The ratio of soluble protein having the correctly expressed tag to total protein isolated gives an indication of the purity of the isolated protein.

A large number of assays are available for measuring total protein in a particular sample. Suitable assays are described below:

https://www.thermofisher.com/uk/en/home/life-science/protein-biology/protein-assays-analysis/protein-assays.html

Assays such the total protein determination assay can be performed on the device, but preferably are performed after removal of the bulk of the expression reagents, which would otherwise dominate measures of total protein. The fluorescence assay for presence of expressed tags can be performed during the process of expression, or can be performed as an end-point by addition of the relevant detector.

Described are assays that measure both purity and levels of soluble yield. Data useful for purification would be both recovered yield of soluble protein (mg/mL) for each purification step and the purity of the recovered protein. The total protein quantification can be performed using an assay such as a Bradford or BCA assay as listed below:

Www dot thermofisher dot com/uk/en/home/life-science/protein-biology/protein-assays-analysis/protein-assays/protein-assay-selection-guide dot html

% Purity=soluble mg/ml*100/total mg/ml

The assays can be performed in parallel in identical droplets, or serially within the same droplets.

In addition to screen for the best conditions for expression, the device can also be used to screen the best conditions for purification of particular proteins. A protein of interest can be made with a variety of different amino acid appendages acting as affinity agents. The expressed amino acids can be exposed to a variety of different beads and the amount of bound material determined. A variety of washing steps can be performed. Thus as well as screening for efficient synthesis, efficient purification conditions can also be identified. Once identified, the optimal conditions and beads can be used for scale-up and purification.

As a hypothetical example, a variety of nucleic acid templates can be screened for expression. The best conditions may give say 20 micrograms of protein having the correct tag. A variety of purification conditions can be screened. The total amount of recovered material having the expressed tags may be say 14 micrograms, hence giving a protein recover of 14/20. However the recovery may give 25 micrograms of total recovered protein, i.e. 13 micrograms of protein which does not contain the correct tag. Thus the purity would be 14/25. More efficient washing could be performed to improve the purity, ideally without lowering the 14 micrograms of correct material.

Such screening, characterising and purification can all be performed on a single digital microfluidic device.

Once the screen is completed, the screening reagents can be moved to a waste area on the device. Alternatively the reagents can be removed entirely from the device. The removal can be performed by exchanging a bulk oil layer under positive flow. The positive flow may come for example using a syringe pump. The flow may be balanced using multiple syringe pumps which input and withdraw fluid at the same rate. Alternatively the device can be angled to enable a flow through an exit hole at the lowest part of the device. Such a flow of fluid may allow the device to become air filled or filled with a washing fluid. Further bulk oil and reagents can be reintroduced to the empty device having no aqueous droplets.

Disclosed herein is a method comprising

a. taking a digital microfluidic device having a planar array of electrodes;
b. using a variety of different conditions to synthesise a protein of interest having a tag in one or more droplets on the device, thereby identifying the optimal conditions for the expression of soluble protein having the tag;
c. removing the droplets from the device;
d. using the optimal conditions to synthesise a protein of interest in a further population of droplets on the device;
e. capturing the proteins via affinity to magnetic beads, thereby immobilising the proteins;
f. moving the droplets using the electrodes, thereby removing the immobilised synthesised proteins from the droplet;
g. washing the immobilised proteins;
h. removing the proteins from the beads by releasing the proteins into further droplets; and
i. measuring the total concentration of protein and the yield of soluble protein in a portion of the droplets to determine the soluble yield and purity of the synthesised protein.

Material recovered from the device can be separate droplets from those that have been used to determine yield and purity. Thus the isolated protein can be obtained without contamination from other quantitation reagents and is ready for further use at a known concentration and purity.

Both expression and purification conditions can be optimised during screening. Disclosed is a method comprising

a. taking a digital microfluidic device having a planar array of electrodes;
b. using a variety of different conditions to synthesise and purify a protein of interest having a tag in one or more droplets on the device, thereby identifying the optimal conditions for the expression and purification of soluble protein having the tag by measuring the total concentration of protein and the yield of soluble protein to determine the soluble yield and purity of the synthesised protein;
c. removing the droplets from the device;
d. using the optimal conditions to synthesise a protein of interest in a further population of droplets on the device;
e. capturing the proteins via affinity to magnetic beads, thereby immobilising the proteins;
f. moving the droplets using the electrodes, thereby removing the immobilised synthesised proteins from the droplet;
g. washing the immobilised proteins; and
h. removing the proteins from the beads by releasing the proteins into further droplets.

The term digital microfluidic device refers to a device having a two-dimensional array of planar microelectrodes. The term excludes any devices simply having droplets in a flow of oil in a channel. The droplets are moved over the surface by electrokinetic forces by activation of particular electrodes. Upon activation of the electrodes the dielectric layer becomes less hydrophobic, thus causing the droplets to spread onto the surface. A digital microfluidic (DMF) device set-up is known in the art, and depends on the substrates used, the electrodes, the configuration of those electrodes, the use of a dielectric material, the thickness of that dielectric material, the hydrophobic layers, and the applied voltage.

The droplets can be aqueous droplets. The droplets can contain an oil immiscible organic solvent such as for example DMSO. The droplets can be a mixture of water and solvent, providing the droplets do not dissolve into the bulk oil.

Digital microfluidics (DMF) refers to a two-dimensional planar surface platform for lab-on-a-chip systems that is based upon the manipulation of microdroplets. Droplets can be dispensed, moved, stored, mixed, reacted, or analyzed on a platform with a set of insulated electrodes. Digital microfluidics can be used together with analytical analysis procedures such as mass spectrometry, colorimetry, electrochemical, and electrochemiluminescense.

The droplet can be moved using any means of electrokinesis. The aqueous droplet can be moved using electrowetting-on-dielectric (EWoD). Electrowetting on a dielectric (EWoD) is a variant of the electrowetting phenomenon that is based on dielectric materials. During EWoD, a droplet of a conducting liquid is placed on a dielectric layer with insulating and hydrophobic properties. Upon activation of the electrodes the dielectric layer becomes less hydrophobic, thus causing the droplet to spread onto the surface.

The electrical signal on the EWoD or optically-activated amorphous silicon (a-Si) EWoD device can be delivered through segmented electrodes, active-matrix thin-film transistors or digital micromirrors. Optically-activated s-Si EWoD devices are well known in the art for actuating droplets (J. Adhes. Sci. Technol., 2012, 26, 1747-1771).

The oil in the device can be any water immiscible liquid. The oil can be mineral oil, silicone oil such as dodecamethylpentasiloxane, an alkyl-based solvent such as decane or dodecane, or a fluorinated oil. The oil can be oxygenated prior to or during the expression process.

The silicone oil can be octamethylcyclotetrasiloxane (CTS), decamethyltetrasiloxane (DMTS) or dodecamethylpentasiloxane (DMPS).

The surfactant in the aqueous layer can be a pluronic surfactant. Pluronic surfactants are also known as poloxamers, and are a class of synthetic block copolymers which consist of hydrophilic poly(ethylene oxide) (PEO) and hydrophobic poly(propylene oxide) (PPO), arranged in an A-B-A triblock structure, thus giving PEO-PPO-PEO. The surfactant may be Pluronic F127.

The pluronic surfactant can be present at less than 0.1%. High levels of surfactant are detrimental to the detection of protein expression. The pluronic concentration can be between 0.025 and 0.1%. The concentration may be 0.05%.

Disclosed is a composition comprising 0.05% w/w Pluronic F127 in an aqueous buffer in a filler fluid of 0.1% span85 in dodecamethylpentasiloxane (DMPS) and use thereof in electrowetting applications, including protein expression.

A source of supplemental oxygen can be supplied to the droplets. For example droplets or gas bubbles containing gaseous or dissolved oxygen can be merged with the aqueous droplets during the protein expression. Alternatively the source of oxygen can be a molecular source which releases oxygen. Alternatively the droplets can be moved to an air/liquid boundary to enable increased diffusion of oxygen from a gaseous environment. Alternatively the oil can be oxygenated.

The droplet can be formed before entering the microfluidic device and flowed into the device. Alternatively the droplets can be merged on the device. Included is a method comprising merging a first droplet containing a nucleic acid template such as a plasmid with a second droplet containing a cell-free system having the components for protein expression to form the droplet.

The droplets can be actuated on a hydrophobic surface on the digital microfluidic device (ACS Nano 2018, 12, 6, 6050-6058). The hydrophobic surface can be a hydrophobic surface such as polytetrafluoroethylene (PTFE), Teflon AF (DuPont Inc), CYTOP (AGC Chemicals Inc), or FluoroPel (Cytonix LLC). The hydrophobic surface may be modified in such a way to reduce biofouling, especially biofouling resulting from exposure to CFPS reagents or nucleic acid reagents. The hydrophobic surface may also be superhydrophobic, such as NeverWet (NeverWet LLC) or Ultra-Ever Dry (Flotech Performance Systems Ltd). Superhydrophobic surfaces prevent biofouling compared with typical fluorocarbon-based hydrophobic surfaces. Superhydrophobic surfaces thus prolong the capability of digital microfluidic devices to move CFPS droplets and general solutions containing biopolymers (RSC Adv., 2017, 7, 49633-49648). The hydrophobic surface can also be a slippery liquid infused porous surface (SLIPS), which can be formed by infusing Krtox-103 oil (DuPont) with porous PTFE film (Lab Chip, 2019, 19, 2275).

For electrowetting on dielectrics (EWoD), the change in contact angle of reagent upon the application of electric potential is an inverse function of surface tension. Thus, for low voltage EWoD operations, reduction in surface tension is achieved by addition of surfactants to reagents, which for CFPS reactions means to the lysate and to the DNA. This results in a dilution of the lysate, and it has been seen, in experiments, that diluting the lysate results in a decrease in expression level of the protein of interest. Thus performing CFPS on DMF where the surfactants are added to the solutions being moved will necessarily result in a dilution of the lysate and thus a decrease in the level of protein expression. In addition to being a problem in its own right, this further complicates extrapolation of on-DMF results to in-tube predictions of protein yield. An additional detriment of having to add surfactants to the samples is that this increases the time required for sample preparation, as well as increasing the potential for inconsistent results due to ‘user error,’ as there is more handling of reagents. An additional detriment of having to add surfactants to the samples is that certain downstream operations are hindered. For example, if a protein of interest is expressed in a cell free system with a GFP₁₁ (or similar) peptide tag, it's downstream complementation with a GFP₁₋₁₀ detector polypeptide is hindered in the presence of surfactant.

Rather than adding surfactants to the aqueous sample, it is instead possible to add surfactant, such as Span85 (sorbitan trioleate), to the oil. This has the advantages of enabling CFPS reactions to proceed on-DMF without dilution or adulteration. Additionally, it simplifies the sample preparation procedure for setting up the reactions, increasing the ease of use and the consistency of results. Using 1% w/w Span85 in dodecane allows for dilution-free CFPS reactions on-DMF, as well as dilution-free detection of the expressed non-fluorescent proteins. Other surfactants besides Span85, and oils other than dodecane could be used. A range of concentrations of Span85 could be used. Surfactants could be nonionic, anionic, cationic, amphoteric. Oils could be mineral oils or synthetic oils, including silicone oils, petroleum oils, and perfluorinated oils. Surfactants can have a detrimental effect on (1) the CFPS reactions and (2) the efficiency of the detection system (if the detection system involves complementation of a tag and detector). For example, by performing the CFPS reaction on-DMF with oil-surfactant mix, the detection of the expressed protein can also proceed without dilution and without adding aqueous surfactant. It has been shown that surfactants reduce the efficiency of some detection systems, including but not limited to the split GFP system, so removing surfactants from the reagent mix and instead adding them to the oil can be beneficial.

Also disclosed is a protein having both a sequence which contains a sub-component of a fluorescent protein and a sequence for affinity purification. The protein may contain GFP₁₁.

The protein may contain a GFP₁₁ peptide amino sequence tag selected from:

(SEQ ID NO: 34)
KRDHMVLLEFVTAAGITGT
(SEQ ID NO: 35)
KRDHMVLHEFVTAAGITGT
(SEQ ID NO: 36)
KRDHMVLHESVNAAGIT
(SEQ ID NO: 37)
RDHMVLHEYVNAAGIT
(SEQ ID NO: 38)
GDAVQIQEHAVAKYFTV
(SEQ ID NO: 39)
GDTVQLQEHAVAKYFTV
(SEQ ID NO: 40)
GETIQLQEHAVAKYFTE

The protein, may contain a GFP₁₁ peptide amino sequence tag selected from:

(SEQ ID NO: 34)
KRDHMVLLEFVTAAGITGT
(SEQ ID NO: 35)
KRDHMVLHEFVTAAGITGT
(SEQ ID NO: 36)
KRDHMVLHESVNAAGIT
(SEQ ID NO: 37)
RDHMVLHEYVNAAGIT
(SEQ ID NO: 38)
GDAVQIQEHAVAKYFTV
(SEQ ID NO: 39)
GDTVQLQEHAVAKYFTV
(SEQ ID NO: 40)
GETIQLQEHAVAKYFTE

in combination with binding tag selected from

Alfa-tag
(SEQ ID NO: 1)
(SRLEEELRRRLTE)
Avi-tag
(SEQ ID NO: 2)
(GLNDIFEAQKIEWHE)
C-tag
(SEQ ID NO: 3)
(EPEA)
Calmodulin-tag
(SEQ ID NO: 4)
(KRRWKKNFIAVSAANRFKKISSSGAL)
Dogtag
(SEQ ID NO: 5)
(DIPATYEFTDGKHYITNEPIPPK)
E-tag
(SEQ ID NO: 6)
(GAPVPYPDPLEPR)
FLAG
(SEQ ID NO: 7)
(DYKDDDDK)
G4T
(SEQ ID NO: 8)
(EELLSKNYHLENEVARLKK)
HA
(SEQ ID NO: 9)
(YPYDVPDYA)
His
(SEQ ID NO: 10)
(HHHHHH)
Isopeptag
(SEQ ID NO: 11)
(TDKDMTITFTNKKDAE)
lanthanide binding tag
(SEQ ID NO: 12)
(LBT)(FIDTNNDGWIEGDELLLEEG)
Myc
(SEQ ID NO: 13)
(EQKLISEEDL)
NE-Tag
(SEQ ID NO: 14)
(TKENPRSNQEESYDDNES)
Poly Glutamate-tag
(SEQ ID NO: 15)
(EEEEEEE)
Poly Arginine-tag
(SEQ ID NO: 16)
(RRRRRRR)
Rho1D4-tag
(SEQ ID NO: 17)
(TETSQVAPA)
SBP-tag
(SEQ ID NO: 18)
(MDEKTTGWRGGHVVEGLAGELEQLRARLEHHPQGQREP)
Sdytag
(SEQ ID NO: 19)
(DPIVMIDNDKPIT)
SH3
(SEQ ID NO: 20)
(STVPVAPPRRRRG)
Snooptag
(SEQ ID NO: 21)
(KLGDIEFIKVNK)
Softag 1
(SEQ ID NO: 22)
(SLAELLNAGLGGS)
Softag 3
(SEQ ID NO: 23)
(TQDPSRVG)
Spot-tag
(SEQ ID NO: 24)
(PDRVRAVSHWSS)
Spytag
(SEQ ID NO: 25)
(AHIVMVDAYKPTK)
S-tag
(SEQ ID NO: 26)
(KETAAAKFERQHMDS)
Strep-tag
(SEQ ID NO: 27)
(WSHPQFEK)
Strep-tag II
(SEQ ID NO: 28)
(AWAHPQPGG)
T7tag
(SEQ ID NO: 29)
(MASMTGGQQMG)
TC-tag
(SEQ ID NO: 30)
(EVHTNQDPLD)
Ty-tag
(SEQ ID NO: 31)
(CCPGCC)
VSV-tag
(SEQ ID NO: 32)
(YTDIEMNRLGK)
Alfa-tag
(SEQ ID NO: 33)
(DLYDDDDK).

The protein may have both a GFP₁₁ region and a His tag. The protein may have both a GFP₁₁ region and a strep-tag or Strep-tag II.

Additionally the protein can have a region for solubility, for example selected from maltose binding protein (MBP), Small Ubiquitin-like Modifier (SUMO), Glutathione S-transferase (GST) or thioredoxin (TRX) or those listed above.

### Devices

The manipulation of droplets by the application of electrical potential can be achieved on electrodes covered with an insulator or a dielectric or a series of insulators or dielectrics. Droplet manipulation as a result of an applied electrical potential is known as electrowetting. Electrokinesis occurs as result of a non-uniform electric field that influences the hydrostatic equilibrium of a dielectric liquid (dielectrophoresis or DEP) or a change in the contact angle of the liquid on solid surface (electrowetting-on-dielectric or EWoD). DEP can also be used to create forces on polarizable particles to induce their movement. The electrical signal can be transmitted to a discrete electrode, a transistor, an array of transistors, or a sheet of semi-conductor film whose electrical properties can be modulated by an optical signal.

EWoD phenomena occur when droplets are actuated between two parallel electrodes covered with a hydrophobic insulator or dielectric. The electric field at the electrode-electrolyte interface induces a change in the surface tension, which results in droplet motion as a result of a change in droplet contact angle. The electrowetting effect can be quantitatively treated using Young-Lippmann equation:

cos θ−cos θ₀=(½γLG)c·V²

where θ₀ is the contact angle when the electric field across the interfacial layer is zero, γLG is the liquid-gas tension, c is the specific capacitance (given as εᵣ. ε₀/t, where εᵣ is dielectric constant of the insulator/dielectric, ε₀ is permittivity of vacuum, t is thickness) and V is the applied voltage or electrical potential. The change in contact angle (inducing droplet movement) is thus a function of surface tension, electrical potential, dielectric thickness, and dielectric constant.

When a droplet is actuated by EWoD, there are two opposing sets of forces that act upon it: an electrowetting force induced by electric field and resistant forces that include the drag forces resulting from the interaction of the droplet with filler medium and the contact line friction (ref). The minimum voltage applied to balance the electrowetting force with the sum of all drag forces (threshold voltage) is variably determined by the thickness-to-dielectric contact ratio of the insulator/dielectric, (t/εᵣ)^{1/2}. Thus, to reduce actuation voltage, it is required to reduce (t/εᵣ)^{1/2} (i.e., increase dielectric constant or decrease insulator/dielectric thickness). To achieve low voltage actuation, thin insulator/dielectric layers must be used. However, the deposition of high quality thin insulator/dielectric layers is a technical challenge, and these thin layers are easily damaged before the desired electrowetting contact angle is large enough to drive the droplet is achieved. Most academic studies thus report the use of much higher voltages>100V on easily fabricated, thick dielectric films (>3 μm) to effect electrowetting.

High voltage EWoD-based devices with thick dielectric films, however, have limited industrial applicability largely due to their limited droplet multiplexing capability. The use of low voltage devices including thin-film transistors (TFT) and optically-activated amorphous silicon layers (a-Si) have paved the way for the industrial adoption of EWoD-based devices due to their greater flexibility in addressing electrical signals in a highly multiplex fashion. The driving voltage for TFTs or optically-activated a-Si are low (typically <15 V). The bottleneck for fabrication and thus adoption of low voltage devices has been the technical challenge of depositing high quality, thin film insulators/dielectrics. Hence there has been a particular need for improving the fabrication and composition of thin film insulator/dielectric devices.

Typically, the electrodes (or the array elements) used for EWoD are covered with (i) a hydrophilic insulator/dielectric and a hydrophobic coating or (ii) a hydrophobic insulator/dielectric. Commonly used hydrophobic coatings comprise of fluoropolymers such as Teflon AF 1600 or CYTOP. The thickness of this material as a hydrophobic coating on the dielectric is typically <100 nm and can have defects in the form of pinholes or a porous structure; hence, it is particularly important that the insulator/dielectric is pinhole free to avoid electrical shorting. Teflon has also been used as an insulator/dielectric, but it has higher voltage requirements due to its low dielectric constant and the thickness required to make it pinhole free. Other hydrophobic insulator/dielectric materials can include polymer-based dielectrics such as those based on siloxane, epoxy (e.g. SU-8), or parylene (e.g., parylene N, parylene C, parylene D, or parylene HT). Due to minimal contact angle hysteresis and a higher contact angle with aqueous solutions, Teflon is still used as a hydrophobic topcoat on these insulator/dielectric polymers. However, there are difficulties in reliably producing <1 micron pinhole-free coatings of parylene or SU-8; thus, the thickness of these materials is typically kept at a 2-5 microns at the cost of increased voltage requirements for electrowetting. It has also been reported that traditional EWoD devices with parylene C are easily broken and unstable for repeated droplet manipulation with cell culture medium. Multi-layer insulator devices deposited with metal-oxide and parylene C films have been used to produce a more robust insulator/dielectric and enable operations with lower applied voltages. Inorganic materials, such metal oxides and semiconductor oxides, commonly used in the CMOS industry as “gate dielectrics”, have been used as insulator/dielectric for EWoD devices. They offer the advantage of utilizing standard cleanroom processes for thin film depositions (<100 nm). These materials are inherently hydrophilic, requiring an additional hydrophobic coating, and can be prone to pinhole formation as a result of thin film layer deposition process. Together with the need for lower voltage operations of EWoD, recent developmental work has focused on (1) using materials with improved dielectric properties (e.g., using high-dielectric constant insulators/dielectrics), (2) optimizing the fabrication process to make the insulator/dielectric pinhole free to avoid dielectric breakdown.

Operation of EWoD devices suffers from contact angle saturation and hysteresis, which is believed to be brought about by either one or combination of these phenomena: (1) entrapment of charges in the hydrophobic film or insulator/dielectric interface, (2) adsorption of ions, (3) thermodynamic contact angle instabilities, (4) dielectric breakdown of dielectric layer, (5) the electrode-electrode-insulator interface capacitance (arising from the double layer effect), and (6) fouling of the surface (such as by biomacromolecules). One of the adverse effects of this hysteresis is reduced operational lifetime of the EWoD-based device.

Contact angle hysteresis is believed to be a result of charge accumulation at the interface or within the hydrophobic insulator after several operations. The required actuation voltage increases due to this charging phenomenon resulting in eventual catastrophic dielectric breakdown. The most probable explanation is that pinholes at the insulator/dielectric may allow the liquid to come into contact with the electrode causing electrolysis. Electrolysis is further facilitated by pinhole-prone or porous hydrophobic insulators.

Most of the studies to understand contact angle hysteresis on EWoD have been conducted on short time scales and with low conductivity solutions. Long duration actuations (e.g., >1 hour) and high conductivity solutions (e.g., 1 M NaCl) could produce several effects other than electrolysis. The ions in solution can permeate through the hydrophobic coat (under the applied electric field) and interact with the underlying insulator/dielectric. Ion permeation can result in (1) change in dielectric constant due to charge entrapment (which is different from interfacial charging) and (2) change in surface potential of a pH sensitive metal oxide. Both can result in reduction of electrowetting forces to manipulate aqueous droplets, leading to contact angle hysteresis. The inventors have previously found that the damage from high conductivity solutions reduces or disables electrowetting on electrodes by inhibiting the modulation of contact angle when an electric field is applied.

An electrokinetic device includes a first substrate having a matrix of electrodes, wherein each of the matrix electrodes is coupled to a thin film transistor, and wherein the matrix electrodes are overcoated with a functional coating comprising: a dielectric layer in contact with the matrix electrodes, a conformal layer in contact with the dielectric layer, and a hydrophobic layer in contact with the conformal layer; a second substrate comprising a top electrode; a spacer disposed between the first substrate and the second substrate and defining an electrokinetic workspace; and a voltage source operatively coupled to the matrix electrodes.

The dielectric layer may comprise silicon dioxide, silicon oxynitride, silicon nitride, hafnium oxide, yttrium oxide, lanthanum oxide, titanium dioxide, aluminium oxide, tantalum oxide, hafnium silicate, zirconium oxide, zirconium silicate, barium titanate, lead zirconate titanate, strontium titanate, or barium strontium titanate. The dielectric layer may be between 10 nm and 100 μm thick. Combinations of more than one material may be used, and the dielectric layer may comprise more than one sublayer that may be of different materials.

The conformal layer may comprise a parylene, a siloxane, or an epoxy. It may be a thin protective parylene coating in between the insulating dielectric and the hydrophobic coating. Typically, parylene is used as a dielectric layer on simple devices. In this invention, the rationale for deposition of parylene is not to improve insulation/dielectric properties such as reduction in pinholes, but rather to act as a conformal layer between the dielectric and hydrophobic layers. The inventors find that parylene, as opposed to other similar insulating coatings of the same thickness such as PDMS (polydimethylsiloxane), prevent contact angle hysteresis caused by high conductivity solutions or solutions deviating from neutral pH for extended hours. The conformal layer may be between 10 nm and 100 μm thick.

The hydrophobic layer may comprise a fluoropolymer coating, fluorinated silane coating, manganese oxide polystyrene nanocomposite, zinc oxide polystyrene nanocomposite, precipitated calcium carbonate, carbon nanotube structure, silica nano-coating, or slippery liquid-infused porous coating.

The elements may comprise one or more of a plurality of array elements, each element containing an element circuit; discrete electrodes; a thin film semiconductor in which the electrical properties can be modulated by incident light; and a thin film photoconductor whose properties can be modulated by incident light.

The functional coating may include a dielectric layer comprising silicon nitride, a conformal layer comprising parylene, and a hydrophobic layer comprising an amorphous fluoropolymer. This has been found to be a particularly advantageous combination.

The electrokinetic device may include a controller to regulate a voltage provided to the individual matrix electrodes. The electrokinetic device may include a plurality of scan lines and a plurality of gate lines, wherein each of the thin film transistors is coupled to a scan line and a gate line, and the plurality of gate lines are operatively connected to the controller. This allows all the individual elements to be individually controlled.

The second substrate may also comprise a second hydrophobic layer disposed on the second electrode. The first and second substrates may be disposed so that the hydrophobic layer and the second hydrophobic layer face each other, thereby defining the electrokinetic workspace between the hydrophobic layers.

The method is particularly suitable for aqueous droplets with a volume of 1 μL or smaller.

The EWoD-based devices shown and described below are active matrix thin film transistor devices containing a thin film dielectric coating with a Teflon hydrophobic top coat. These devices are based on devices described in the E Ink Corp patent filing on “Digital microfluidic devices including dual substrate with thin-film transistors and capacitive sensing”, US patent application no 2019/0111433, incorporated herein by reference.

An exemplary method may include the following steps:

1. Express the proteins bearing a purification tag (e.g. His, Strep) using cell free lysates on a (TFT-based) digital microfluidic platform.
2. Contact a droplet containing protein expressed in a cell free lysate with superparamagnetic beads bearing a purification moiety (e.g. Ni-NTA for His tag, Streptavidin for Strep tag) and incubate for a period of time to enable the tag and purification moiety to interact.
3. Apply a magnetic field (e.g. by bringing a magnet into proximity with the digital microfluidic array or turning on an electromagnet) to pellet the superparamagnetic particles.
4. Remove the supernatant droplet from the pelleted superparamagnetic particles using EWoD forces and then wash the superparamagnetic particles by passing over droplets of an appropriate composition and then removing those droplets. The superparamagnetic particles may be resuspended in the wash droplets and then re-pelleted prior to removal of the wash droplets; this wash process may be repeated multiple times, which may increase the protein purity obtained.
5. Elute the tagged protein from the purification moiety on the superparamagnetic particles by contacting them with a drop containing an elution solution (e.g. imidazole for His tag/Ni NTA or desthiobiotin for Strep tag/Streptavidin).
6. Pellet the superparamagnetic particles and move the drop containing the purified protein to a reaction zone for downstream processing on the digital microfluidic array OR move the drop to a collection port to be retrieved from the digital microfluidic array.

### EXAMPLES

1. Express a His/Strep tagged protein in an E coli lysate on a DMF array by contacting a double stranded DNA construct with an E coli cell free lysate and incubating on the array (circa 4-12 hours, for example 6 hours), at a controlled temperature (circa 20-37° C., for example 29° C.) with mixing
a. this may be premixed off the array
b. this may be mixed on the array
   2. Add a solution of SPMP to the DMF array—dispense drop(s) and move to a purification zone
   3. Apply magnetic field (for example using an automated magnetic platform, or alternatively using a manual magnet or turning on an electromagnet) to pellet the SPMP
   4. Move the supernatant drop to waste
   5. Apply a wash drop, resuspend SPMP, pellet SPMP, remove wash drop to waste
   6. Optionally repeat step (5) from one to five times
   7. Apply an elution drop, resuspend SPMP, pellet SPMP, remove elution drop to collection port
   8. Retrieve elution drop and analyse by gel electrophoresis/blot/other analytical technique as appropriate The purity achieved can be greater than 70% using His tag and greater than 90% using Strep tag.

### Screen:

### Screening Conditions for Optimal Expression Conditions:

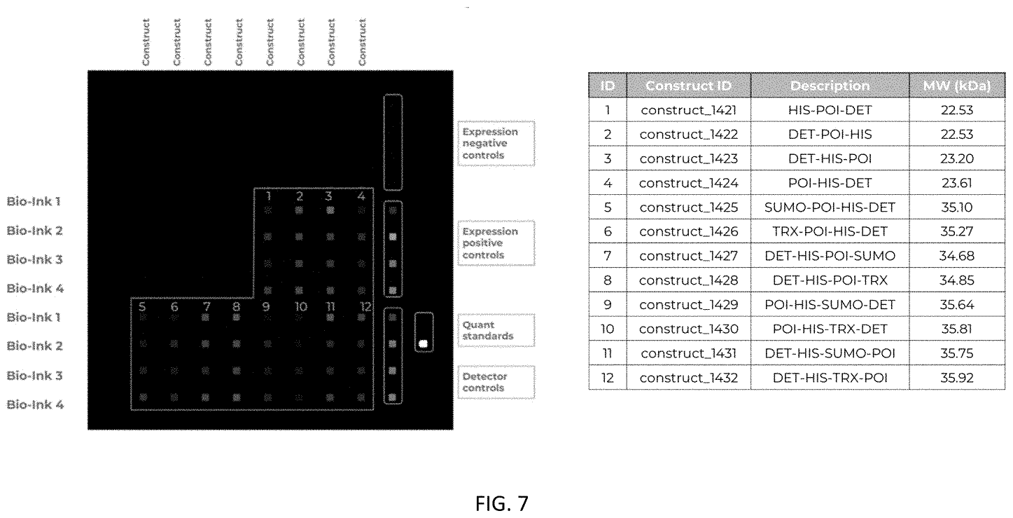

An active-matrix TFT cartridge device was loaded onto a Nuclera eprotein instrument and filled with a base fluid containing 0.1% span85 in dodecamethylpentasiloxane (DMPS). Screen reagents (12 nucleic acid constructs and 4 expression reagents) were loaded into ports of the device and droplets dispensed and mixed in order to express protein (POI) in 48 separate conditions. Expressed proteins all contain a ccGFP₁₁ tag in order to identify the expression using fluorescent complementation with a ccGFP₁₋₁₀ protein present as detector species. The optimal nucleic acid sequence and expression system for a given protein can be identified by monitoring the fluorescence signal generated by the assembled GFP₁₋₁₁. Fluorescence and control data can be seen in FIG. 7.

Once the best expression conditions are identified and stored, the device can be transitioned and used again to scale up the protein synthesis using the identified optimal conditions. The screen droplets are actuated close to an exit port and removed from the device using a flow of fluid whilst the aqueous droplets are no longer held by electrode actuation. The removal of screening population, together with the basefluid is achieved by angling the device and applying a negative pressure via a syringe to enable flow through and collection of the reagents via the exit hole at the lowest part of the device. Further bulk phase oil and aqueous reagents are introduced to the device for the scale up ‘print’ operation.

### Print Example 1

On device His-tag purification of ccGFP protein in vitro expressed in Print BioInk 1 (an E coli lysate).

An active-matrix TFT cartridge device was loaded onto a Nuclera eprotein instrument and filled with a base fluid containing 0.1% span85 in dodecamethylpentasiloxane (DMPS). Print reagents were loaded into ports of the device as shown in Table 1, in 2×11.2 μL aliquots, using a 20 μL 8-channel multi-pipette. Magbead solution was prepared by aspiration of storage buffer, washing with, and resuspending in an equivalent volume of Magbead Bind Buffer.

TABLE 1
Reagent loading summary

| | | | | | Droplet |
|---|---|---|---|---|---|
| | | Reagent | | μL/ | volume/ |
| Port | Reagent | conc | | port | px² |
| C01 | Print Wash Buffer | 1X | | 22.4 | 100 |
| C02-04 | Magbeads in Magbead Bind | 40 | mg/mL | 22.4 | 49 |
| | Buffer | | | | |
| C05 | Construct_1986 DNA solution | 24 | nM | 22.4 | 16 |

| | | | | |
|---|---|---|---|---|
| C06-07 | Print BioInk 1 | 1.33 X | 22.4 | 49 |
| C08 | Print Elution Buffer | 1X | 22.4 | 100 |

Electrowetting on dielectric (EWoD) was employed to generate a 96-reaction zone print droplet array by dispensing and merging LEC DNA with BioInk reagent. In-vitro transcription translation was allowed to proceed for 10 hours at 29° C. Droplets were moved periodically during incubation to ensure mixing. Following incubation, print expression reaction droplets were merged 1:1 with magbead droplets and mixed. Magbead suspensions were incubated at 29° C. for up to 16 minutes to allow proteins to bind to the magbeads. Three magbead suspension droplets were merged to form a 32-droplet array and moved to the magnetic pelletting zone on the device above a magneto-thermal module (MTM). Elevating the MTM caused half of the 32-droplet magbead array to pellet magbeads into 16 discrete pellets. Pelletting was conducted for 70 seconds to minimize magbead loss. Depleted expression reagents were aspirated away from the magbead pellet by EWoD. Wash buffer droplets were moved over the magbead pellets and the MTM was lowered, allowing the magbead pellet to be resuspended in the wash buffer and moved away from the magnetic pelleting zones. The steps above were then repeated with the remaining 16 magbead suspension droplets, resulting in the formation of a 32-droplet array of washed magbead suspension droplets.

The washed magbead droplets were moved to the pelleting zones on the device above the MTM using EWoD. Elevating the MTM caused the half of the 32-droplet magbead array to pellet magbeads into 16 discrete pellets. Pelleting was conducted for 70 seconds to minimize magbead loss. Wash buffer was aspirated away from the magbead pellet by EWoD. Print elution buffer droplets were moved over the washed magbead pellets and the MTM was lowered, allowing the washed magbead pellet to be resuspended in the Print Elution Buffer and moved away from the magnetic pelleting zones. The steps above were then repeated with the remaining 16 magbead suspension droplets, resulting in the formation of a 32-droplet array of Print Elution magbead suspension droplets.

Magbeads were incubated in the Print Elution Buffer as a magbead suspension for 12 minutes at 29° C. to maximize elution of bound expressed protein from the magbeads into solution.

After incubation, Print Elution Buffer magbead suspension droplets were moved to the pelleting zones on the device above the MTM using EWoD. Elevating the MTM caused half of the 32-droplet magbead array to pellet magbeads into 16 discrete pellets. Pelletting was conducted for 70 seconds to minimize magbead loss. Eluted protein in Print Elution Buffer was aspirated away from the magbead pellet by EWoD whilst the MTM was in its elevated position, to hold the magbeads in place. The elution steps above were then repeated with the remaining 16 Print Elution buffer magbead suspension droplets.

The 32 aspirated purified protein elution droplets were moved to the edge of the device and merged into a single pool using EWoD. This single pool was then harvested from the device by manual pipetting.

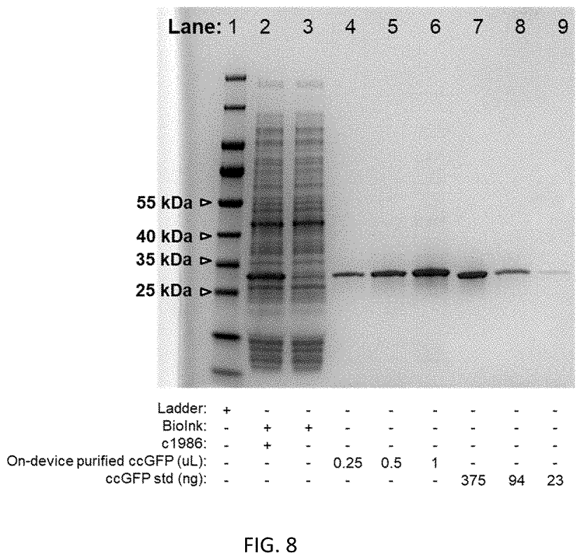

Harvested purified protein was analyzed by SDS-PAGE (BioRad mini protean 4-15% Mini-PROTEAN TGX Precast Gel) in parallel with control samples, as outlined in Table 2. Samples were mixed with SDS-loading buffer and heat denatured before running through the gel. A typical gel image is shown in FIG. 8. Expressed ccGFP (from construct_1986) runs as a 27 kDa band on the gel, coincident to recombinant ccGFP standard protein, expressed in a cell-based expression system and purified independently, by conventional IMAC, on an AKTA protein purification system.

TABLE 2
SDS-PAGE loading conditions

| Lane | Sample | μL/lane |
|---|---|---|
| 1 | Molecular weight ladder (Thermo PAGE ruler plus | 2.5 |
| | prestained mwt ladder) | |
| 2 | BioInk 1 positive control crude expression reaction | 4 |
| | (plus DNA)* | |
| 3 | BioInk 1 negative control (no DNA)* | 4 |
| 4 | Harvested, device-purified expressed ccGFP** | 0.25 |
| 5 | Harvested, device-purified expressed ccGFP** | 0.5 |
| 6 | Harvested, device-purified expressed ccGFP** | 1 |
| 7 | Recombinant ccGFP standard (94 ng/μL in loading | 4 |
| | buffer) | |
| 8 | Recombinant ccGFP standard (23 ng/μL in loading | 4 |
| | buffer) | |
| 9 | Recombinant ccGFP standard (5.8 ng/μL in loading | 4 |
| | buffer) | |

*0.1X dilution in loading buffer;
**0.25X dilution in loading buffer

### Print Example 2

On device His-tag purification of ccGFP protein in vitro expressed in Print BioInk 2 (a reconstituted transcription/translation system).

An active-matrix TFT cartridge device was loaded onto a Nuclera eprotein instrument and filled with a base fluid containing 0.1% span85 in dodecamethylpentasiloxane (DMPS). Print reagents were loaded into ports of the device as shown in Table 3, in 2×11.2 μL aliquots, using a 20 μL 8-channel multi-pipette. Magbead solution was prepared by aspiration of storage buffer, washing with, and resuspending in an equivalent volume of Magbead Bind Buffer.

TABLE 3
Reagent loading summary

| | | | | | Droplet |
|---|---|---|---|---|---|
| | | Reagent | | μL/ | volume/ |
| Port | Reagent | conc | | port | px² |
| C01 | Print Wash Buffer | 1X | | 22.4 | 100 |
| C02-04 | Magbeads in Magbead Bind | 40 | mg/mL | 22.4 | 49 |
| | Buffer | | | | |
| C05 | Construct_1986 DNA | 24 | nM | 22.4 | 16 |
| | solution | | | | |

| | | | | |
|---|---|---|---|---|
| C06-07 | Print BioInk 2 | 1.33 X | 22.4 | 49 |
| C08 | Print Elution Buffer | 1X | 22.4 | 100 |

Electrowetting on dielectric (EWoD) was employed to generate a 96-reaction zone print droplet array by dispensing and merging LEC DNA with BioInk reagent. In-vitro transcription translation was allowed to proceed for 10 hours at 29° C. Droplets were moved periodically during incubation to ensure mixing. Following incubation, print expression reaction droplets were merged 1:1 with magbead droplets and mixed. Magbead suspensions were incubated at 29° C. for up to 16 minutes to allow proteins to bind to the magbeads. Three magbead suspension droplets were merged to form a 32-droplet array and moved to the magnetic pelleting zone on the device above the magneto-thermal module (MTM). Elevating the MTM caused the half of the 32-droplet magbead array to pellet magbeads into 16 discrete pellets. Pelleting was conducted for 70 seconds to minimize magbead loss. Depleted expression reagents were aspirated away from the magbead pellet by EWoD. Wash buffer droplets were moved over the magbead pellets and the MTM was lowered, allowing the magbead pellet to be resuspended in the wash buffer and moved away from the magnetic pelleting zones. The steps above were then repeated with the remaining 16 magbead suspension droplets, resulting in the formation of a 32-droplet array of washed magbead suspension droplets.

The washed magbead droplets were moved to the pelletting zones on the device above the MTM using EWoD. Elevating the MTM caused the half of the 32-droplet magbead array to pellet magbeads into 16 discrete pellets. Pelleting was conducted for 70 seconds to minimize magbead loss. Wash buffer was aspirated away from the magbead pellet by EWoD. Print elution buffer droplets were moved over the washed magbead pellets and the MTM was lowered, allowing the washed magbead pellet to be resuspended in the Print Elution Buffer and moved away from the magnetic pelleting zones. The steps above were then repeated with the remaining 16 magbead suspension droplets, resulting in the formation of a 32-droplet array of Print Elution magbead suspension droplets.

Magbeads were incubated in the Print Elution Buffer as a magbead suspension for 12 minutes at 29° C. to maximize elution of bound expressed protein from the magbeads into solution.

After incubation, Print Elution Buffer magbead suspension droplets were moved to the pelleting zones on the device above the MTM using EWoD. Elevating the MTM caused half of the 32-droplet magbead array to pellet magbeads into 16 discrete pellets. Pelleting was conducted for 70 seconds to minimize magbead loss. Eluted protein in Print Elution Buffer was aspirated away from the magbead pellet by EWoD whilst the MTM was in its elevated position, to hold the magbeads in place. The elution steps above were then repeated with the remaining 16 Print Elution buffer magbead suspension droplets.

The 32 aspirated eluted protein droplets were moved to the edge of the device and merged into a single pool using EWoD. This single pool was then harvested from the device by manual pipetting.

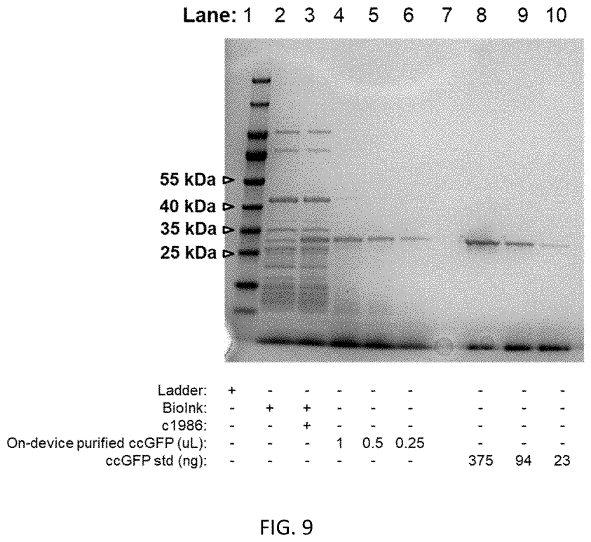

Harvested purified protein was analyzed by SDS-PAGE (BioRad mini protean 4-15% Mini-PROTEAN TGX Precast Gel) in parallel with control samples, as outlined in Table 4. Samples were mixed with SDS-loading buffer and heat denatured before running through the gel. A typical gel image is shown in FIG. 9. Expressed ccGFP (from construct_1986) runs as a 27 kDa band on the gel, coincident to recombinant ccGFP expressed in a cell-based expression system and purified independently, by conventional IMAC, on an AKTA bio protein purification system.

TABLE 4
SDS-PAGE loading conditions

| | | μL/ |
|---|---|---|
| Lane | Sample | lane |
| 1 | Molecular weight ladder (Thermo PAGE ruler plus | 2.5 |
| | prestained mwt ladder) | |
| 2 | Harvested, device-purified expressed ccGFP** | 1 |
| 3 | Harvested, device-purified expressed ccGFP** | 0.5 |
| 4 | Harvested, device-purified expressed ccGFP** | 0.25 |
| 5 | Recombinant ccGFP standard (94 ng/μL in loading buffer) | 4 |
| 6 | Recombinant ccGFP standard (23 ng/μL in loading buffer) | 4 |
| 7 | Recombinant ccGFP standard (5.8 ng/μL in loading buffer) | 4 |

*0.1X dilution in loading buffer;
**0.25X dilution in loading buffer

### Print Example 3

On device Strep-tag purification of ccGFP protein in vitro expressed in Print BioInk 2 (a reconstituted transcription/translation system).

An active-matrix TFT cartridge device was loaded onto a Nuclera eprotein instrument and filled with a base fluid containing 0.1% span85 in dodecamethylpentasiloxane (DMPS). Print reagents were loaded into ports of the device as shown in Table 5, in 2×11.2 μL aliquots, using a 20 μL 8-channel multi-pipette. Strep-Magbead solution was prepared by aspiration of storage buffer, washing with, and resuspending in a minimum volume of Strep-Wash buffer.

TABLE 5
Reagent loading summary

| | | | | Droplet |
|---|---|---|---|---|
| | | Reagent | μL/ | volume/ |
| Port | Reagent | conc | port | px² |
| C01 | Strep-wash Buffer | 1X | 22.4 | 100 |
| C02-04 | Strep-Magbeads in Strep-wash | 5X | 22.4 | 49 |
| | Buffer | | | |
| C05 | Construct_1669 DNA solution | 24 nM | 22.4 | 16 |
| C06-07 | Print BioInk 2 | 1.33 X | 22.4 | 49 |
| C08 | Strep-elution Buffer | 1X | 22.4 | 100 |

Electrowetting on dielectric (EWoD) was employed to generate a 96-reaction zone print droplet array by dispensing and merging LEC DNA with BioInk reagent. In-vitro transcription translation was allowed to proceed for 10 hours at 29° C. Droplets were moved periodically during incubation to ensure mixing. Following incubation, print expression reaction droplets were merged 1:1 with magbead droplets and mixed. Magbead suspensions were incubated at 29° C. for up to 16 minutes to allow proteins to bind to the magbeads. Three magbead suspension droplets were merged to form a 32-droplet array and moved to the magnetic pelleting zone on the device, directly below the position of a manual magnet array (MMA) placed manually on the top-plate of the device. Placing the MMA in position on the device caused half of the 32-droplet magbead array to pellet magbeads into 16 discrete pellets.

Pelleting was conducted for 70 seconds to minimize magbead loss. Depleted expression reagents were aspirated away from the magbead pellet by EWoD. Strep-wash buffer droplets were moved over the magbead pellets and the MMA was removed from the device, allowing the magbead pellet to be resuspended in the strep-wash buffer and moved away from the magnetic pelleting zones. The steps above were then repeated with the remaining 16 magbead suspension droplets, resulting in the formation of a 32-droplet array of washed magbead suspension droplets.

The washed magbead droplets were moved to the pelleting zones on the device directly below the position of an MMA placed manually on the top-plate of the device, using EWoD. Placing the MMA in position on the device caused the half of the 32-droplet magbead array to pellet magbeads into 16 discrete pellets. Pelleting was conducted for 70 seconds to minimize magbead loss. Strep-wash buffer was aspirated away from the magbead pellet by EWoD. Strep-elution buffer droplets were moved over the washed magbead pellets and the MMA was removed from the device, allowing the washed magbead pellet to be resuspended in the Strep-elution Buffer and moved away from the magnetic peletting zones. The steps above were then repeated with the remaining 16 magbead suspension droplets, resulting in the formation of a 32-droplet array of Strep-elution magbead suspension droplets.

Magbeads were incubated in the Strep-elution Buffer as a magbead suspension for 12 minutes at 29° C. to maximize elution of bound expressed protein from the magbeads into solution.

After incubation, Strep-elution Buffer magbead suspension droplets were moved to the pelleting zones on the device, directly below the position of a manual magnet array (MMA) placed manually on the top-plate of the device, using EWoD.

Placing the MMA in position on the device caused half of the 32-droplet magbead array to pellet magbeads into 16 discrete pellets. Pelleting was conducted for 70 seconds to minimize magbead loss. Eluted protein in Strep-elution Buffer was aspirated away from the magbead pellet by EWoD whilst the MMA was in position on the device, to hold the magbeads in place. The elution steps above were then repeated with the remaining 16 Strep-elution buffer magbead suspension droplets.

The 32 aspirated purified protein elution droplets were moved to the edge of the device and merged into a single pool using EWoD. This single pool was then harvested from the device by manual pipetting.

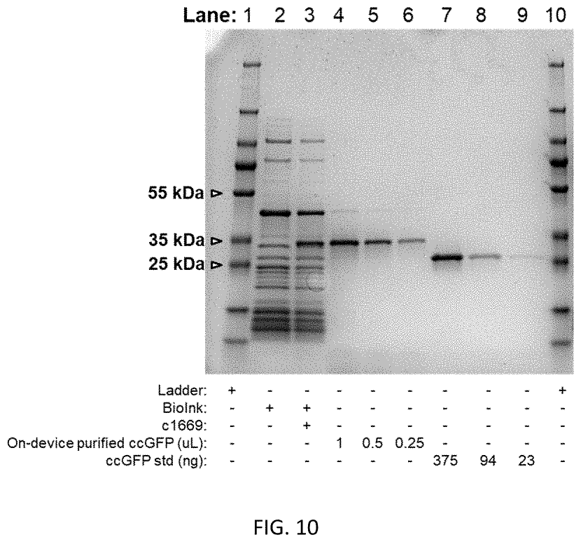

Harvested purified protein was analyzed by SDS-PAGE (BioRad mini protean 4-15% Mini-PROTEAN TGX Precast Gel) in parallel with control samples, as outlined in Table 6. Samples were mixed with SDS-loading buffer and heat denatured before running through the gel. A typical gel image is shown in FIG. 10. Expressed ccGFP (from construct_1669) runs as a 27 kDa band on the gel, coincident to recombinant ccGFP standard protein, expressed in a cell-based expression system and purified independently, by conventional IMAC, on an AKTA protein purification system.

TABLE 6
SDS-PAGE loading conditions

| Lane | Sample | μL/lane |
|---|---|---|
| 1 | Molecular weight ladder (Thermo PAGE ruler plus | 2.5 |
| | prestained mwt ladder) | |
| 2 | BioInk 2 negative control (no DNA)* | 4 |
| 3 | BioInk 2 positive control crude expression reaction | 4 |
| | (plus DNA)* | |
| 4 | Harvested, device-purified expressed ccGFP** | 1 |
| 5 | Harvested, device-purified expressed ccGFP** | 0.5 |
| 6 | Harvested, device-purified expressed ccGFP** | 0.25 |
| 7 | Recombinant ccGFP standard (94 ng/μL in loading | 4 |
| | buffer) | |
| 8 | Recombinant ccGFP standard (23 ng/μL in loading | 4 |
| | buffer) | |
| 9 | Recombinant ccGFP standard (5.8 ng/μL in loading | 4 |
| | buffer) | |

*0.1X dilution in loading buffer;
**0.25X dilution in loading buffer

1. A method for protein synthesis comprising
a. taking a digital microfluidic device having a planar array of electrodes;
b. synthesising a protein of interest having a binding tag in droplets on the device;
c. capturing the proteins via the binding tags, thereby immobilising the proteins;
d. moving the droplets using the electrodes, thereby removing the synthesised proteins from the droplet;
e. optionally washing the immobilised proteins; and
f. optionally releasing the proteins into further droplets.

2. The method according to claim 1 comprising;
a. taking a digital microfluidic device having a planar array of electrodes;
b. using a variety of different conditions to synthesise a protein of interest having a tag in one or more droplets on the device, thereby identifying the optimal conditions for the expression of soluble protein having the tag;
c. removing the droplets from the device;
d. using the optimal conditions to synthesise a protein of interest in a further population of droplets on the device;
e. capturing the proteins via affinity to magnetic beads, thereby immobilising the proteins;
f. moving the droplets using the electrodes, thereby removing the immobilised synthesised proteins from the droplet;
g. optionally washing the immobilised proteins; and
h. removing the proteins from the device, either by releasing the proteins into further droplets and removing the protein droplets from the device or by removing the magnetic beads from the device.

3. The method according to claim 1, wherein the immobilised protein is released into further droplets for removal from the device.

4. The method according to claim 1, wherein protein synthesis is performed using cell free lysates.

5. The method according to claim 1, wherein protein synthesis performed using assembled components for transcription and translation in a system of purified recombinant elements (PURE).

6. The method according to claim 1, wherein multiple proteins of interest are synthesised in parallel on the device.

7. The method according to claim 1, wherein the yield of soluble protein is determined by fluorescence complementation.

8. The method according to claim 7, wherein the expressed proteins comprise GFP₁₁.

9. The method according to claim 1, wherein the binding tags and detection tags are the same.

10. The method according to claim 1, wherein affinity purification uses binding tags selected from:
Alfa-tag
(SRLEEELRRRLTE)
Avi-tag
(GLNDIFEAQKIEWHE)
C-tag
(EPEA)
Calmodulin-tag
(KRRWKKNFIAVSAANRFKKISSSGAL)
Dogtag
(DIPATYEFTDGKHYITNEPIPPK)
E-tag
(GAPVPYPDPLEPR)
FLAG
(DYKDDDDK)
G4T
(EELLSKNYHLENEVARLKK)
HA
(YPYDVPDYA)
His
(HHHHHH)
Isopeptag
(TDKDMTITFTNKKDAE)
lanthanide binding tag
(LBT)
(FIDTNNDGWIEGDELLLEEG)
Myc
(EQKLISEEDL)
NE-Tag
(TKENPRSNQEESYDDNES)
Poly Glutamate-tag
(EEEEEEE)
Poly Arginine-tag
(RRRRRRR)
Rho1D4-tag
(TETSQVAPA)
SBP-tag
(MDEKTTGWRGGHVVEGLAGELEQLRARLEHHPQGQREP)
Sdytag
(DPIVMIDNDKPIT)
SH3
(STVPVAPPRRRRG)
Snooptag
(KLGDIEFIKVNK)
Softag 1
(SLAELLNAGLGGS)
Softag 3
(TQDPSRVG)
Spot-tag
(PDRVRAVSHWSS)
Spytag
(AHIVMVDAYKPTK)
S-tag
(KETAAAKFERQHMDS)
Strep-tag
(WSHPQFEK)
Strep-tag II
(AWAHPQPGG)
T7tag
(MASMTGGQQMG)
TC-tag
(EVHTNQDPLD)
Ty-tag
(CCPGCC)
VSV-tag
(YTDIEMNRLGK)
Xpress-tag
(DLYDDDDK).

11. The method according to claim 1, wherein additional droplets are passed over the immobilised proteins and removed, further purifying the proteins.

12. The method according to claim 1, wherein an assay to determine the total amount of purified protein is performed.

13. The method according to claim 12, wherein the assay uses Coomassie.

14. The method according to claim 1, wherein the digital microfluidic device comprises an oil-filled or humidified gaseous environment, wherein the humidified gaseous environment is achieved by enclosing or sealing the digital microfluidic device and providing on-board reagent reservoirs.

15. The method according to claim 14, wherein the oil is mineral oil, silicone oil, an alkyl-based solvent, or a fluorinated oil, wherein the oil optionally contains a surfactant.

16. (canceled)

17. The method according to claim 1, wherein the screening step identifies the optimal conditions for purification on the device.

18-19. (canceled)

20. The method according to claim 1 comprising
a. taking a digital microfluidic device having a planar array of electrodes;
b. using a variety of different conditions to synthesise and purify a protein of interest having a tag in one or more droplets on the device, thereby identifying the optimal conditions for the expression and purification of soluble protein having the tag by measuring the total concentration of purified protein and the yield of soluble protein to determine the soluble yield and purity of the synthesised protein;
c. removing the droplets from the device;
d. using the optimal conditions to synthesise a protein of interest in a further population of droplets on the device;
e. capturing the proteins via affinity to magnetic beads, thereby immobilising the proteins;
f. moving the droplets using the electrodes, thereby removing the immobilised synthesised proteins from the droplets;
g. washing the immobilised proteins; and
h. removing the proteins from the beads by releasing the proteins into further droplets.

21. (canceled)

22. The method according to claim 1 wherein the expressed protein in step b contains a GFP₁₁ peptide amino sequence tag selected from:
KRDHMVLLEFVTAAGITGT
KRDHMVLHEFVTAAGITGT
KRDHMVLHESVNAAGIT
RDHMVLHEYVNAAGIT
GDAVQIQEHAVAKYFTV
GDTVQLQEHAVAKYFTV
GETIQLQEHAVAKYFTE
and a binding tag selected from
Alfa-tag (SRLEEELRRRLTE)
Avi-tag (GLNDIFEAQKIEWHE)
C-tag (EPEA)
Calmodulin-tag (KRRWKKNFIAVSAANRFKKISSSGAL)
Dogtag (DIPATYEFTDGKHYITNEPIPPK)
E-tag (GAPVPYPDPLEPR)
FLAG (DYKDDDDK)
G4T (EELLSKNYHLENEVARLKK)
HA (YPYDVPDYA)
His (HHHHHH)
Isopeptag (TDKDMTITFTNKKDAE)
lanthanide binding tag (LBT)
(FIDTNNDGWIEGDELLLEEG)
Myc (EQKLISEEDL)
NE-Tag (TKENPRSNQEESYDDNES)
Poly Glutamate-tag (EEEEEEE)
Poly Arginine-tag (RRRRRRR)
RholD4-tag (TETSQVAPA)
SBP-tag (MDEKTTGWRGGHVVEGLAGE
LEQLRARLEHHPQGQREP)
Sdytag (DPIVMIDNDKPIT)
SH3 (STVPVAPPRRRRG)
Snooptag (KLGDIEFIKVNK)
Softag 1 (SLAELLNAGLGGS)
Softag 3 (TQDPSRVG)
Spot-tag (PDRVRAVSHWSS)
Spytag (AHIVMVDAYKPTK)
S-tag (KETAAAKFERQHMDS)
Strep-tag (WSHPQFEK)
Strep-tag II (AWAHPQPGG)
T7tag (MASMTGGQQMG)
TC-tag (EVHTNQDPLD)
Ty-tag (CCPGCC)
VSV-tag (YTDIEMNRLGK)
Xpress-tag (DLYDDDDK).

23. The method according to claim 1 for synthesizing a protein of interest (POI) comprising:
a. taking a digital microfluidic device having a planar array of electrodes;
b. synthesising a protein of interest having a GFP₁₁ binding tag and a protease cleavage site in droplets on the device;
c. capturing the POI via the binding tags to form GFP₁₋₁₁, and capturing the GFP₁₋₁₁, thereby immobilising the POI,
d. moving the droplets using the electrodes, thereby removing the synthesised fluorescent proteins from the droplet;
e. optionally washing the immobilised fluorescent proteins; and
f. releasing the proteins of interest into further droplets using a protease.

24. The method according to claim 22, wherein the GFP₁₋₁₁ is immobilised via a binding tag attached to GFP₁₋₁₀ wherein the GFP₁₋₁₀ contains a binding tag selected from:
Alfa-tag (SRLEEELRRRLTE)
Avi-tag (GLNDIFEAQKIEWHE)
C-tag (EPEA)
Calmodulin-tag (KRRWKKNFIAVSAANRFKKISSSGAL)
Dogtag (DIPATYEFTDGKHYITNEPIPPK)
E-tag (GAPVPYPDPLEPR)
FLAG (DYKDDDDK)
G4T (EELLSKNYHLENEVARLKK)
HA (YPYDVPDYA)
His (HHHHHH)
Isopeptag (TDKDMTITFTNKKDAE)
lanthanide binding tag (LBT)
(FIDTNNDGWIEGDELLLEEG)
Myc (EQKLISEEDL)
NE-Tag (TKENPRSNQEESYDDNES)
Poly Glutamate-tag (EEEEEEE)
Poly Arginine-tag (RRRRRRR)
RholD4-tag (TETSQVAPA)
SBP-tag(MDEKTTGWRGGHVVEGLAG
ELEQLRARLEHHPQGQREP)
Sdytag (DPIVMIDNDKPIT)
SH3 (STVPVAPPRRRRG)
Snooptag (KLGDIEFIKVNK)
Softag 1 (SLAELLNAGLGGS)
Softag 3 (TQDPSRVG)
Spot-tag (PDRVRAVSHWSS)
Spytag (AHIVMVDAYKPTK)
S-tag (KETAAAKFERQHMDS)
Strep-tag (WSHPQFEK)
Strep-tag II (AWAHPQPGG)
T7tag (MASMTGGQQMG)
TC-tag (EVHTNQDPLD)
Ty-tag (CCPGCC)
VSV-tag (YTDIEMNRLGK)
Xpress-tag (DLYDDDDK).


SEQUENCE LISTING (40 sequences)

SEQ ID NO: 1 - protein, 13 aa, synthetic construct
  REGION 1..13: note = Synthetic: Alfa-tag
SRLEEELRRRLTE

SEQ ID NO: 2 - protein, 15 aa, synthetic construct
  REGION 1..15: note = Synthetic: Avi-tag
GLNDIFEAQKIEWHE

SEQ ID NO: 3 - protein, 4 aa, synthetic construct
  REGION 1..4: note = Synthetic: C-tag
EPEA

SEQ ID NO: 4 - protein, 26 aa, synthetic construct
  REGION 1..26: note = Synthetic: Calmodulin-tag
KRRWKKNFIAVSAANRFKKISSSGAL

SEQ ID NO: 5 - protein, 23 aa, synthetic construct
  REGION 1..23: note = Synthetic: Dogtag
DIPATYEFTDGKHYITNEPIPPK

SEQ ID NO: 6 - protein, 13 aa, synthetic construct
  REGION 1..13: note = Synthetic: E-tag
GAPVPYPDPLEPR

SEQ ID NO: 7 - protein, 8 aa, synthetic construct
  REGION 1..8: note = Synthetic: FLAG
DYKDDDDK

SEQ ID NO: 8 - protein, 19 aa, synthetic construct
  REGION 1..19: note = Synthetic: G4T
EELLSKNYHLENEVARLKK

SEQ ID NO: 9 - protein, 9 aa, synthetic construct
  REGION 1..9: note = Synthetic: HA
YPYDVPDYA

SEQ ID NO: 10 - protein, 6 aa, synthetic construct
  REGION 1..6: note = Synthetic: His
HHHHHH

SEQ ID NO: 11 - protein, 16 aa, synthetic construct
  REGION 1..16: note = Synthetic: Isopeptag
TDKDMTITFTNKKDAE

SEQ ID NO: 12 - protein, 20 aa, synthetic construct
  REGION 1..20: note = Synthetic: lanthanide binding tag (LBT)
FIDTNNDGWIEGDELLLEEG

SEQ ID NO: 13 - protein, 10 aa, synthetic construct
  REGION 1..10: note = Synthetic: Myc
EQKLISEEDL

SEQ ID NO: 14 - protein, 18 aa, synthetic construct
  REGION 1..18: note = Synthetic: NE-Tag
TKENPRSNQEESYDDNES

SEQ ID NO: 15 - protein, 7 aa, synthetic construct
  REGION 1..7: note = Synthetic: Poly Glutamate-tag
EEEEEEE

SEQ ID NO: 16 - protein, 7 aa, synthetic construct
  REGION 1..7: note = Synthetic: Poly Arginine-tag
RRRRRRR

SEQ ID NO: 17 - protein, 9 aa, synthetic construct
  REGION 1..9: note = Synthetic: Rho1D4-tag
TETSQVAPA

SEQ ID NO: 18 - protein, 38 aa, synthetic construct
  REGION 1..38: note = Synthetic: SBP-tag
MDEKTTGWRGGHVVEGLAGELEQLRARLEHHPQGQREP

SEQ ID NO: 19 - protein, 13 aa, synthetic construct
  REGION 1..13: note = Synthetic: Sdytag
DPIVMIDNDKPIT

SEQ ID NO: 20 - protein, 13 aa, synthetic construct
  REGION 1..13: note = Synthetic: SH3
STVPVAPPRRRRG

SEQ ID NO: 21 - protein, 12 aa, synthetic construct
  REGION 1..12: note = Synthetic: Snooptag
KLGDIEFIKVNK

SEQ ID NO: 22 - protein, 13 aa, synthetic construct
  REGION 1..13: note = Synthetic: Softag 1
SLAELLNAGLGGS

SEQ ID NO: 23 - protein, 8 aa, synthetic construct
  REGION 1..8: note = Synthetic: Softag 3
TQDPSRVG

SEQ ID NO: 24 - protein, 12 aa, synthetic construct
  REGION 1..12: note = Synthetic: Spot-tag
PDRVRAVSHWSS

SEQ ID NO: 25 - protein, 13 aa, synthetic construct
  REGION 1..13: note = Synthetic: Spytag
AHIVMVDAYKPTK

SEQ ID NO: 26 - protein, 15 aa, synthetic construct
  REGION 1..15: note = Synthetic: S-tag
KETAAAKFERQHMDS

SEQ ID NO: 27 - protein, 8 aa, synthetic construct
  REGION 1..8: note = Synthetic: Strep-tag
WSHPQFEK

SEQ ID NO: 28 - protein, 9 aa, synthetic construct
  REGION 1..9: note = Synthetic: Strep-tag II
AWAHPQPGG

SEQ ID NO: 29 - protein, 11 aa, synthetic construct
  REGION 1..11: note = Synthetic: T7tag
MASMTGGQQMG

SEQ ID NO: 30 - protein, 10 aa, synthetic construct
  REGION 1..10: note = Synthetic: TC-tag
EVHTNQDPLD

SEQ ID NO: 31 - protein, 6 aa, synthetic construct
  REGION 1..6: note = Synthetic: Ty-tag
CCPGCC

SEQ ID NO: 32 - protein, 11 aa, synthetic construct
  REGION 1..11: note = Synthetic: VSV-tag
YTDIEMNRLGK

SEQ ID NO: 33 - protein, 8 aa, synthetic construct
  REGION 1..8: note = Synthetic: Xpress-tag
DLYDDDDK

SEQ ID NO: 34 - protein, 19 aa, synthetic construct
  REGION 1..19: note = Synthetic: complementary GFP11 peptide amino acid sequence tag
KRDHMVLLEFVTAAGITGT

SEQ ID NO: 35 - protein, 19 aa, synthetic construct
  REGION 1..19: note = Synthetic: complementary GFP11 peptide amino acid sequence tag
KRDHMVLHEFVTAAGITGT

SEQ ID NO: 36 - protein, 17 aa, synthetic construct
  REGION 1..17: note = Synthetic: complementary GFP11 peptide amino acid sequence tag
KRDHMVLHESVNAAGIT

SEQ ID NO: 37 - protein, 16 aa, synthetic construct
  REGION 1..16: note = Synthetic: complementary GFP11 peptide amino acid sequence tag
RDHMVLHEYVNAAGIT

SEQ ID NO: 38 - protein, 17 aa, synthetic construct
  REGION 1..17: note = Synthetic: complementary GFP11 peptide amino acid sequence tag
GDAVQIQEHAVAKYFTV

SEQ ID NO: 39 - protein, 17 aa, synthetic construct
  REGION 1..17: note = Synthetic: complementary GFP11 peptide amino acid sequence tag
GDTVQLQEHAVAKYFTV

SEQ ID NO: 40 - protein, 17 aa, synthetic construct
  REGION 1..17: note = Synthetic: complementary GFP11 peptide amino acid sequence tag
GETIQLQEHAVAKYFTE